(19) United States
(12) Patent Application Publication
(10) Pub. No.: US 2010/0292299 A1
(43) Pub. Date: Nov. 18, 2010

(54) Nucleotide Motifs Providing Localization Elements and Methods of Use

(76) Inventors: Joshua T. Mendell, Baltimore, MD (US); Hun-Way Wang, Baltimore, MD (US); Erik A. Wentzel, Millersville, MD (US)

Correspondence Address: EDWARDS ANGELL PALMER & DODGE LLP, P.O. BOX 55874, BOSTON, MA, 02205, US

(21) Appl. No.: 12/308,811

(22) PCT Filed: Jun. 20, 2007
(86) PCT No.: PCT/US07/14444

§ 371 Date: Sep. 15, 2009

Related U.S. Application Data
(60) Provisional application No. 60/815,090, filed on Jun. 20, 2006.

(51) Int. Cl. A61K 31/7105; C07H 21/02; C12N 15/63; A61P 43/00

(52) U.S. Cl. 514/44 A; 536/23.1; 536/24.5; 514/44 R; 435/320.1

(57) ABSTRACT

The instant invention describes nucleotide motifs comprising a localization element and small RNA molecules comprising the localization motifs, and methods of use in gene silencing. The nucleotide motifs have use in preventing and treating diseases or disorders.

### RELATED APPLICATIONS

This application claims the benefit of U.S. Provisional Application No.: 60/815,090, filed Jun. 20, 2006, the entire contents of which is expressly incorporated herein by reference.

### BACKGROUND OF THE INVENTION

Small RNA molecules are common and effective modulators of gene expression in a variety of eukaryotic cells. Small ribonucleic acid (RNA) molecules are short RNA sequences that are produced by nearly all eukaryotes (e.g., fungi, plants, and animals). However, rather than encoding a protein, small RNAs function to reduce the mRNA abundance or protein abundance of the gene which is the “target.” In certain instances small RNAs can also result in target gene regulation by affecting chromatin structure. As such, noncoding small RNAs have emerged as important regulators of gene expression at both transcriptional and posttranscriptional levels. Commonly known types of small RNAs include small interfering RNAs (siRNAs), microRNAs (miRNAs), Piwi-interacting RNAs (piRNAs) and repeat-associated small interfering RNAs (rasiRNAs).

One obstacle in small RNA design is the stability of the RNA construct. In vivo applications of siRNA or microRNA may require molecules with enhanced stability. In particular, therapeutic application of siRNA and miRNA gene silencing will benefit from improvements in small RNA molecule stability and specificity.

Thus, there is a need in the art for the design small RNAs with increased stability, or small RNAs that are highly enriched in the nucleus. Small RNAs that are highly enriched in the nucleus may prove useful for the manipulation of nuclear steps in gene expression, and for use in the prevention and treatment of disease.

### SUMMARY OF THE INVENTION

The instant invention is based on the discovery of a sequence motif that promotes nuclear localization, nuclear import and nuclear accumulation. The invention is based on the finding that the human microRNA miR-29b, in contrast to other studied animal miRNAs, is predominantly localized to the nucleus. The distinctive hexanucleotide terminal motif of miR-29b was found to act as a transferable nuclear localization element that directs nuclear enrichment of small RNAs to which it is attached. As such, the sequence motif provides a sequence that is useful to design effective small RNAs.

Accordingly, in one aspect, the invention features an isolated nucleotide motif comprising a localization element for an RNA or protein.

In one embodiment, the nucleotide motif comprises a localization element, where the localization element is attached to the 3′ terminus of the RNA or protein. The localization element can comprise any number of nucleotides. In a particular embodiment, the nucleotide motif comprises a localization element, where the localization element is a hexanucleotide motif.

In another embodiment, the nucleotide motif comprises a localization element, where the localization element directs nuclear localization of the RNA or protein. In another embodiment, the nucleotide motif comprises a localization element, where the localization element promotes nuclear import of the RNA or protein. In another embodiment, the nucleotide motif comprises a localization element, where the localization element increases nuclear accumulation of the RNA. In another embodiment, the nucleotide motif comprises a localization element, where the localization element silences expression of the RNA target sequence.

In a particular embodiment, the nucleotide motif of any of the above-mentioned aspects comprises a localization element where the RNA is a small RNA.

In another embodiment, the nucleotide motif comprises a localization element where the small RNA is selected from a small interfering RNA (siRNA), a microRNA (miRNA), a Piwi-interacting RNA (piRNA), or a repeat associated siRNA (rasiRNA).

In another particular embodiment, the small RNA is modified. In one embodiment, the modification is a chemical modification. In another embodiment, the modification comprises linking the small RNA to an agent. In a further embodiment, the agent is selected from antibodies, aptamers or cholesterol.

In one embodiment, the nucleotide motif comprises a localization element where the localization element is selected from the group consisting of: SEQ ID NO: 1 (AGUGUU), SEQ ID NO: 2 (UGUGUU), SEQ ID NO: 3 (ACUGUU), SEQ ID NO: 4 (AGAGUU), SEQ ID NO: 5 (AGUCUU), SEQ ID NO: 6 (AGUGAU) and SEQ ID NO: 7 (AGUGUA).

In another embodiment, the nucleotide motif comprises a localization element where the localization element comprises SEQ ID NO: 8 (AGNGUN), where N is any nucleotide.

In another aspect, the invention features an isolated RNA molecule comprising a first segment comprising a nucleotide motif comprising a localization element, and a second segment comprising a region of nucleotides adjacent to the localization element.

In one embodiment, the first segment and the second segment comprise a double-stranded region of nucleotides about 15-80 nucleotides in length. In another embodiment, the nucleotide motif comprising a localization element is adjacent to the 3′ terminus of the second segment.

In a further embodiment, the second segment double-stranded region of nucleotides adjacent to the first segment double-stranded region of nucleotides provides specificity for the RNA to a target gene. In another embodiment, the nucleotide motif comprising a localization element is located at the 3′ terminus of the double-stranded nucleotide region. In yet a further embodiment, the first segment and the second segment comprising a double-stranded region of nucleotides are annealed such that there is a 2-nucleotide 3′ overhang.

In another embodiment, the RNA is a small RNA. In a further embodiment, the small RNA is selected from a small interfering RNA (siRNA), a microRNA (miRNA), a Piwi-interacting RNA (piRNA), or a repeat associated siRNA (rasiRNA).

In a particular embodiment, the small RNA is modified. In another particular embodiment, the modification is a chemical modification. In still a further embodiment, the modification comprises linking the small RNA to an agent. In a particular embodiment, the agent is selected from antibodies, aptamers, or cholesterol.

In another embodiment, the second segment comprises a region of about 15-80 nucleotides in length comprising a sense RNA strand and an antisense RNA strand that forms an RNA duplex that is approximately 20-80 nucleotides in length. In a further embodiment, the sense and antisense RNA strands that form an RNA duplex are covalently linked by a single-stranded hairpin.

In another particular embodiment, the RNA molecule is targeted to any stretch of approximately 20-35 contiguous nucleotides in a target mRNA sequence.

In another aspect, the invention features an isolated small RNA molecule comprising a first and second segment comprising a sense RNA strand and an antisense RNA strand, wherein the sense and the antisense RNA strands form an RNA duplex, and wherein the small RNA molecule is targeted to any stretch of, approximately 20-35 contiguous nucleotides in a target mRNA sequence, and wherein the first segment comprises a nucleotide motif comprising a localization element.

In one embodiment, the second segment comprises a sense RNA strand and an antisense RNA strand that is about 15-80 nucleotides in length adjacent to the nucleotide sequence. In a further embodiment, the siRNA or miRNA molecule comprises two strands, and at least 1 strand has a 3′ overhang of about 1 to about 6 nucleotides in length.

In another embodiment, the small RNA is selected from a small interfering RNA (siRNA), a microRNA (miRNA), a Piwi-interacting RNA (piRNA), or a repeat associated siRNA (rasiRNA).

In one embodiment of the above-mentioned aspect, the localization element is selected from the group consisting of: SEQ ID NO: 1 (AGUGUU), SEQ ID NO: 2 (UGUGUU), SEQ ID NO: 3 (ACUGUU), SEQ ID NO: 4 (AGAGUU), SEQ ID NO: 5 (AGUCUU), SEQ ID NO: 6 (AGUGAU) and SEQ ID NO: 7 (AGUGUA).

In another embodiment of the above-mentioned aspect, the localization element comprises SEQ ID NO: 8 (AGNGUN), where N is any nucleotide.

In one embodiment of the above-mentioned aspect, the RNA molecule or the small RNA molecule is capable of inhibiting the expression of a target gene in a cell. In a further embodiment, the gene silencing is transcriptional gene silencing. In another embodiment, the gene silencing is post-translational gene silencing. In another particular embodiment, the target gene comprises one or more of an endogenous cellular gene, an exogenous gene or a viral gene.

In one embodiment, the target gene is of mammalian origin. In a particular embodiment, the target gene is of human origin.

In another embodiment, the target gene is expressed in a phenotypically normal cell. In a further embodiment, the target gene is expressed in a diseased cell.

In one particular embodiment, the target gene activity is increased in the cell.

In one embodiment of the above-mentioned aspect, the expression of the target gene is inhibited by at least 50%, 60%, 70%, 75%, 80%, 85%, 90%, 95%, or more by the small inhibitory RNA molecule.

In one embodiment of the above-mentioned aspect, the RNA molecule or the small RNA molecule directs nuclear localization of the RNA.

In a further embodiment of the above-mentioned aspect, the RNA molecule or the small RNA molecule enhances nuclear localization of the RNA.

In still a further embodiment of the above-mentioned aspect, the RNA molecule or the small RNA molecule increases the nuclear accumulation of the RNA.

In another aspect, the invention features a method for inhibiting the expression of a target gene in a cell comprising introducing into the cell an effective amount of an RNA molecule a small RNA molecule according to the aspects as described herein.

In another aspect, the invention features a method for introducing an RNA molecule that silences expression of a target sequence into a cell comprising contacting the cell with an RNA molecule a small RNA molecule according the aspects as described herein.

In another aspect, the invention features method for preventing or treating a disease or disorder, comprising, administering to a subject in need thereof a pharmaceutical composition comprising a therapeutically effective amount of an RNA molecule according to claim 16 or a small RNA molecule according to the aspects as described herein.

In one embodiment, a beneficial therapeutic effect of treating the disease or disorder comprises silencing of at least one gene. In another embodiment, the RNA molecule is in an expression vector.

In a further embodiment, the RNA molecule attenuates expression of a target gene within a cell ex vivo. In still a further embodiment, the RNA molecule attenuates expression of a target gene within a cell in vivo.

In one embodiment, the RNA molecule is administered systemically. In a particular embodiment, the RNA molecule is in a carrier system. In yet a further embodiment, the carrier system is selected from the group consisting of a nucleic acid-lipid particle, a liposome, a micelle, a virosome, a nucleic acid complex, and a mixture thereof.

In another aspect, the invention features a method of making an RNA molecule with enhanced nuclear localization and with specificity to a target gene, comprising obtaining a first segment comprising a nucleotide motif comprising a localization element, and a second segment comprising a region nucleotides that provides specificity to a target gene, wherein the first segment is attached to the 3′ terminus of the second segment, and thereby generating a RNA molecule with enhanced nuclear localization and specificity to a target gene.

In still another aspect the invention features a method of making a RNA molecule with enhanced nuclear import and specificity to a target gene comprising obtaining a first segment comprising a nucleotide motif, and a second segment comprising a region of nucleotides that provides specificity to a target gene, wherein the first segment is attached to the 3′ terminus of the second segment, and thereby generating an RNA molecule with enhanced nuclear import and specificity to a target gene.

In another aspect the invention features a method of making an RNA molecule with increased nuclear accumulation and specificity to a target gene comprising obtaining a first segment comprising a nucleotide motif comprising a localization element, and a second segment comprising a region of nucleotides that provides specificity to a target gene, wherein the first segment is attached to the 3′ terminus of the second segment, and thereby generating a RNA molecule with increased nuclear accumulation and specificity to a target gene.

In still another aspect the invention features a method of making a protein with enhanced nuclear localization, import and accumulation, comprising obtaining a first segment comprising a nucleotide motif comprising a localization element, and a second segment comprising a region of amino acids encoding a protein of interest, wherein the first segment is attached to the region of amino acids, and thereby generating a protein with enhanced nuclear localization, import and accumulation.

In one embodiment, the first segment is attached to the second segment with a linker. In another embodiment, the second segment further comprises a drug or an agent.

In a particular embodiment, the first and second segments comprise a double-stranded region of nucleotides about 15-80 nucleotides in length.

In another particular embodiment, the RNA molecule is capable of silencing expression of the target sequence. In one embodiment, the silencing is transcriptional silencing. In another embodiment, the silencing is post-translational silencing.

In another aspect, the invention features a pharmaceutical composition comprising as an active ingredient an RNA molecule or the small RNA as described in the aspects herein, and a pharmaceutically acceptable carrier.

In one embodiment, the invention features an expression vector capable of coding for an RNA molecule or the small RNA as described in the aspects herein

In another particular embodiment, the pharmaceutical composition comprises the vector.

In one embodiment, the composition is suitable for enteral administration. In a further embodiment, enteral administration is selected from oral administration, rectal administration, and intranasal administration.

In another embodiment, the pharmaceutical composition is suitable for parenteral administration. In a further embodiment, a parenteral administration route is selected from intravascular administration, subcutaneous injection, subcutaneous infusion, direct application (for instance near a site of interest), and inhalation. In a particular embodiment, the direct application is selected from a catheter, an implant, or a pump.

In one embodiment of any of the above-mentioned aspects, the nucleotide motif comprises a localization element that is a hexanucleotide motif.

In another aspect, the invention features a kit comprising the nucleotide motif comprising a localization element of any one of the above-mentioned aspects, and instructions for use.

In another aspect, the invention features a kit comprising the RNA molecule of any one of the above-mentioned aspects, and instructions for use.

In another aspect, the invention features a kit comprising the pharmaceutical composition of any one of the above-mentioned aspects, and instructions for use.

### DESCRIPTION OF THE DRAWINGS

FIGS. 1(A & B) shows validation of a Northern blot assay that can distinguish between the three human miR-29 paralogs. (A) shows sequences of the three human miR-29 paralogs a, b, and c, indicated as SEQ ID NO: 9, SEQ ID NO: 10 and SEQ ID NO; 11, respectively. (B) shows specificity of the Northern blot assay. In the Northern assay, RNA oligos identical to each miR-29 family member were hybridized with probes specific for each miRNA. Less than 10% cross hybridization was observed for the miR-29c probe and the other probes did not detectably cross-hybridize.

FIG. 2(A-D) shows that miR-29b is degraded rapidly in cycling cells but is stable in mitotic cells. (A) shows the results of Northern blot demonstrating discordant expression of miR-29a and miR-29b during the cell cycle. miR-29c (not shown) was not detectable. miR-30, a constitutively expressed miRNA, served as a loading control. Relative expression levels are indicated below the two panels. (B) shows that the miR-29b-1/miR-29a cluster but not the miR-29b-2/miR-29c cluster is expressed in HeLa cells. The genomic organization of each cluster is shown in the schematic on the left (arrows represent primers). Genomic DNA (gDNA) or RNA from cells treated with siRNA directed against Drosha or Luc was amplified. 7SK RNA served as a positive control. (C) shows the results of RT-PCR assay for Drosha processing. Numbers in the schematic indicate amplicons. Primer pairs, indicated by arrows in the schematic and by numbers to the left of the gels, were used to amplify gDNA or RNA from cells treated with siRNA directed against Drosha or Luc. (ID) shows the results of pulse-chase assay to measure synthetic miRNA stability. Northern blotting was used to measure the abundance of si-miR-29a and si-miR-29b at the indicated time points after transfection of RNA duplexes. The mean and SD of calculated half-lives (t½) from three experiments are shown on the right of each blot. The mitotic half-lives were longer than the time course and are therefore reported as >12 hours.

FIG. 3 shows the results of a Northern blot demonstrating that synthetic miR-29b duplexes accumulate in mitotic cells. HeLa cells were transfected with the indicated concentration of RNA duplexes and allowed to continue cycling or were arrested in mitosis. miR-30 served as loading control.

FIG. 4 shows the results of a Northern blot showing that endogenous strand selection bias is preserved in the si-miR-29b duplex. The Northern blot shows that the guide strand of the si-miR-29b duplex is more abundant than the passenger strand in immunopurified RISC. HeLa cells were transfected with si-miR-29b alone (Mock) or in combination with plasmids that express HA-tagged human Argonaute 1 (HA-hAgo1 ) or Argonaute 2 (HA-hAgo2). Immunoprecipitation (IP) with anti-HA antibodies was then performed and the abundance of both strands in the IP fraction was measured. 10% of the total input was loaded on the blots shown on the left.

FIG. 5(A-D) shows that accumulation of miR-29b occurs specifically in mitotic cells. (A) is a Northern blot demonstrating accumulation of miR-29b only in mitotic cells despite prolonged exposure to nocodazole. HeLa cells were synchronized by double-thymidine block, released into nocodazole-containing media, and harvested at the time-points indicated. Both floating and adherent cells were harvested for each time-point except for M phase, which represents a pure population of detached mitotic cells harvested 13 hours after release. A significant fraction of cells had entered mitosis by the 12 hour time-point. (B) shows that within a population of cells, miR-29b accumulates specifically in floating mitotic cells, not in non-mitotic adherent cells. Here, the experiment was performed as in (A) except RNA was isolated separately from floating and adherent cells 13 hours after release. (C) shows a Northern blot demonstrating accumulation of miR-29b in mitotic cells arrested with microtubule poisons that have diverse effects on microtubule dynamics. Cells were treated with the indicated drugs or DMSO (vehicle) for 13 hours and floating cells were harvested. (D) shows that accumulation of miR-29b is not a secondary consequence of apoptosis. Cells were treated with the indicated drugs for 12 hours prior to isolating RNA. In parallel, the fraction of cells entering apoptosis was determined by Annexin V staining and flow cytometry.

FIGS. 6(A & B) shows that miR-29b is imported into the nucleus. (A) is a Northern blot demonstrating the relative nuclear and cytoplasmic abundance of endogenous miR-29a, miR-29b, and miR-21. U6 small nuclear RNA (U6 snRNA) and lysine-tRNA (tRNAlys) served as controls for subcellular fractionation. (B) shows confocal microscopy demonstrating intranuclear localization of a fraction of si-miR-29b but not si-miR-29a (upper panels). The dashed line indicates the nuclear periphery, as defined by 4′,6′-diamidino-2-phenylindole staining (lower panels).

FIG. 7 illustrates that the miR-29b 3′ terminal motif is a transferable nuclear import element. Northern blotting was used to determine the nuclear and cytoplasmic abundance of a series of synthetic siRNAs. All membranes were reprobed with U6 snRNA and lysine-tRNA to verify successful subcellular fractionation (representative blots shown). nt, nucleotide. Highlighted letters in the sequences indicate substitutions or altered positions.

FIGS. 8(A & B) shows plasmid-based expression of miR-29b. (A) shows a Northern blot demonstrating high expression of plasmid-derived miR-29b. Cells were mock transfected or transfected with the pcDNA3.1/miR-29b expression plasmid. U6 snRNA served as a loading control. (B) shows nuclear-cytoplasmic fractionation of cells transfected with the miR-29b expression plasmid or with a plasmid expressing a variant of miR-29b harboring a G-to-C mutation at position 21 (miR-29b(m4), sequence shown in FIG. 10). Whereas plasmid-derived miR-29b was predominantly nuclear, the mutant miRNA was predominantly cytoplasmic, consistent with the behavior of a synthetic si-miRNA duplex harboring the same mutation (FIG. 10). Membranes were re-probed for U6 snRNA and lysine-tRNA to verify successful subcellular fractionation (representative blots shown).

FIGS. 9(A & B) shows mitotic accumulation and nuclear enrichment of miR-291b in murine cells. (A) is a Northern blot demonstrating that miR-29b accumulates in mitotic NIH 3T3 cells relative to unsynchronized cells. Mitotic cells were obtained by nocodazole treatment for 16 hours. U6 snRNA served as loading control. (B) is a Northern blot demonstrating that miR-29b is enriched in the nuclear fraction of NIH 3T3 cells compared to miR-29a. The membrane was re-probed for U6 snRNA and lysine-tRNA to verify successful subcellular fractionation.

FIG. 10 shows nuclear import of miR-29b variants with altered position or altered sequence of the 3′ terminal motif. Northern blotting was used to determine the nuclear and cytoplasmic abundance of a series of synthetic miR-29b variants in which the 3′ terminal motif was moved into the body of the miRNA by one or two nucleotides [si-miR-29b(TM-1) and si-miR-29b(TM-2)] or single-nucleotide mutations were introduced [si-miR-29b(m1) through si-miR-29b(m6)]. Similar results were obtained from 3 independent experiments. Representative blots are shown. All membranes were re-probed with U6 snRNA and lysine-tRNA to verify successful subcellular fractionation.

FIG. 11 shows that the miR-29b 3′ terminal motif does not promote accelerated decay. Northern blotting was used to measure the abundance of synthetic RNAs at the indicated time-points following transfection of RNA duplexes. The mean and standard deviation of calculated half-lives from 2 experiments is shown on the right of each blot.

FIG. 12 shows that the guide and passenger strands of the si-miR-29b duplex exhibit distinct subcellular localizations. The Figure shows a Northern Blot demonstrating that the guide strand of the si-miR-29b duplex is predominantly nuclear while the passenger strand is predominantly cytoplasmic.

### DETAILED DESCRIPTION OF THE INVENTION

The instant invention is based on the discovery that RNAs, for example short-interfering RNAs (siRNAs) microRNAs, (miRNAs), can be tagged with a sequence motif that promotes their nuclear localization, nuclear import and nuclear accumulation. In particular preferred embodiments the sequence motif is a hexanucleotide sequence motif that is located at the 3′ termini.

The present invention discloses the finding that this sequence motif that promotes nuclear localization, nuclear import and nuclear accumulation provides a sequence that is useful to design effective small RNAs (i.e. siRNAs,miRNAs, piRNAs, or rasiRNAs). This novel 3′ terminal sequence enables a more universal design of appropriate small molecule RNAs, and when combined with unique sequences present adjacent to the consensus sequence, constitutes a molecule that has a consensus part (enabling easy design) and a unique part (enabling specific gene silencing).

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. As used herein, the term “adjacent” is meant to refer to the position of the localization element in relation to the second segment comprising a region of nucleotides. In preferred embodiments, adjacent refers to the position of the localization element as 3′ to the second segment comprising a region of nucleotides.

As used herein, by the term “attenuates” is meant that the activity of a gene expression product or level of RNAs or equivalent RNAs encoding one or more gene products is reduced or inhibited below that observed in the absence of the nucleic acid molecule of the invention.

As used herein, the term “cell” is meant to refer to a eukaryotic cell. Typically, the cell is of animal origin and can be a stem cell or somatic cells. Suitable cells can be of, for example, mammalian, avian or plant origin. Examples of mammalian cells include human, bovine, ovine, porcine, murine, and rabbit cells. The cell can be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions).

As used herein the term “effective amount” or “therapeutically effective amount” of an RNA molecule, for example a small RNA molecule such as an siRNA or miRNA molecule, is an amount sufficient to produce the desired effect, e.g., an inhibition of expression of a target sequence in comparison to the normal expression level detected in the absence of the siRNA. Inhibition of expression of a target gene or target sequence is achieved when the value obtained with the siRNA relative to the control is about 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

As used herein the term “expression” is meant to refer to transcription of a gene sequence and, as appropriate, translation of the resulting mRNA transcript to a protein.

As used herein, the term “gene” refers to a nucleic acid (e.g., DNA or RNA) sequence that comprises partial length or entire length coding sequences necessary for the production of a polypeptide or precursor polypeptide.

As used herein, the term “isolated” means altered or removed from the natural state through human intervention. For example, an RNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

As used herein, the term “localization element” is meant to refer to a sequence of nucleotides that directs nuclear enrichment of nucleotides, proteins or other molecules to which it is attached. In certain embodiments, the localization element is 4, 5, 6, 7, 8, 9, 10 or more nucleotides in length. In preferred embodiments, the localization element is 5 nucleotides in length.

As used herein, the term “mammal” is meant to refer to any mammalian species such as a human, mouse, or rat.

As used herein, the term “nuclear accumulation” refers to an increase in the amount or level of a measurable product, for example gene expression or protein level, in the nucleus.

As used herein, the term “nuclear localization” refers to the detection of expression, for example gene expression or protein level, in the nucleus. Nuclear localization refers to enrichment of expression in the nuclear compartment.

As used herein, the term “nuclear import” refers to transport across nuclear membranes.

As used herein, the term “nucleotide motif” refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA may be in the form of siRNA, mRNA, tRNA, rRNA, tRNA, vRNA, and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the term “phenotypically normal cell” refers to a cell that is functioning normally. In certain embodiments, a phenotypically normal cell may not display a diseased phenotype, but may produced increased levels of a gene product, for example a hormone or a signaling protein. In certain preferred embodiments, gene silencing occurs in phenotypically normal cells.

As used herein, the term “Piwi-interacting RNA (piRNA)” is meant to refer to a class of small RNA molecules that are expressed in mammalian testes. Piwi proteins are involved in RNA silencing.

As used herein, the term “repeat-associated small interfering RNAs (rasiRNAs)” is meant to refer to a class of small RNA molecules that arise mainly from the antisense strand.

As used herein, the term “silencing” is meant to refer to the suppression or inhibition of expression of the (target) gene. The term silencing is not necessarily meant to imply reduction of transcription, because gene silencing is believed to operate in at least some cases post-transcriptionally. The degree of gene silencing can be complete so as to abolish production of the encoded gene product. Complete gene silencing can produce a null phenotype. The degree of gene silencing can be partial, with some degree of expression remaining. Partial gene silencing can produce an intermediate phenotype.

As used herein, the terms “small inhibitory RNA (siRNA)” or “micro RNA (miRNA)” are used interchangeably. The terms are meant to include RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 20-24, 21-22, or 21-23 base pairs in length). In preferred embodiments, the terms are meant to include inhibitory or interfering sequences. MiRNA or siRNA duplexes may comprise 3′ overhangs of about 1 to about 4 nucleotides or about 2 to about 3 nucleotides and 5′ phosphate termini. Examples include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

In preferred embodiments, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 30-50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

As used herein, the term “small RNA” is meant to refer to sequences of ribonucleotides that act as repressors of gene expression in plants, animals and many fungi. Small regulatory RNAs are generated via processing of longer double-stranded RNA (dsRNA) precursors and are double stranded. In preferred embodiments, small inhibitory RNAs are between 20-80 nucleotides in length. Small RNAs can control gene expression through transcriptional or post-translational mechanisms, or through targeting epigenetic modifications to specific regions of the genome. Examples of small RNAs include, but are not limited to siRNAs, miRNAs, piRNAs, rasiRNAs, small temporal RNAs, heterochromatic siRNAs, tiny noncoding RNAs,

As used herein, the term “systemic delivery,” refers to delivery that leads to a broad biodistribution of a compound such as an siRNA within an organism. Some techniques of administration can lead to the systemic delivery of certain compounds, but not others. Systemic delivery means that a useful, preferably therapeutic, amount of a compound is exposed to most parts of the body. To obtain broad biodistribution generally requires a blood lifetime such that the compound is not rapidly degraded or cleared (such as by first pass organs (liver, lung, etc.) or by rapid, nonspecific cell binding) before reaching a disease site distal to the site of administration. Systemic delivery of nucleic acid-lipid particles can be by any means known in the art including, for example, intravenous, subcutaneous, and intraperitoneal. In a preferred embodiment, systemic delivery of nucleic acid-lipid particles is by intravenous delivery.

As used herein, the term “local delivery,” refers to delivery of a compound such as an siRNA directly to a target site within an organism. For example, a compound can be locally delivered by direct injection into a disease site such as a tumor or other target site such as a site of inflammation or a target organ such as the liver, heart, pancreas, kidney, and the like.

As used herein, the term “vector” refers to the plasmid, virus or phage chromosome used in cloning to carry the cloned DNA segment. Vectors capable of directing the expression of genes to which they are operatively linked are referred to herein as “expression vectors”. Another type of vector is a genomic integrated vector, or “integrated vector”, which can become integrated into the chromosomal DNA of the host cell. Another type of vector is an episomal vector, i.e., a nucleic acid capable of extra-chromosomal expression. In the present specification, “plasmid” and “vector” are used interchangeably unless otherwise clear from the context

### Nucleotide Motifs

The instant invention is based on the discovery that specific small RNAs contain sequence elements that control their behavior, for example posttranscriptional behavior. These novel sequence elements, or motifs, may provide control of gene expression at the posttranscriptional level, such as subcellular localization, mRNA splicing, 3′ end formation or transport. The instant invention includes the finding that miR-29b, in exemplary embodiments human miR-29b, in contrast to other studied animal miRNAs, is predominantly localized to the nucleus. In preferred embodiments, human miR-29b corresponds to GenBank Accession No. AJ421751.

The instant invention encompasses nucleotide motif comprising a localization element for an RNA or protein. The RNA the RNA is selected from a small interfering RNA (siRNA) or a microRNA (miRNA). In certain examples, the localization element is attached to the 3′ terminus of the RNA or protein. The localization element can be of any number of nucleotides as long as it provides the desired function, for example subcellular localization to the nucleus. As such, the localization element can be 3, 4, 5, 6, 7, 8, 9, Or more nucleotides in length. In preferred embodiments, the localization element is a hexanucleotide motif.

The nucleotide motif comprising a localization element in certain examples may direct nuclear localization of the RNA, enhance nuclear localization of the RNA, or increase the nuclear accumulation of the RNA, or any combination of the above.

The nucleotide motif comprising a localization element of the instant invention, in certain preferred embodiment, silences expression of the RNA target sequence(s). It is to be understood by one of skill in the art that gene silencing is meant to refer to the suppression or inhibition of expression of a target gene. The term silencing is not necessarily meant to imply reduction of transcription, because gene silencing is believed to operate in at least some cases post-transcriptionally. The degree of gene silencing can be complete so as to abolish production of the encoded gene product. Complete gene silencing can produce a null phenotype. The degree of gene silencing can be partial, with some degree of expression remaining. Partial gene silencing can produce an intermediate phenotype.

In certain examples, the localization element is selected from the group consisting of: SEQ ID NO: 1 (AGUGUU), SEQ ID NO: 2 (UGUGUU), SEQ ID NO: 3 (ACUGUU), SEQ ID NO: 4 (AGAGUU), SEQ ID NO: 5 (AGUCUU), SEQ ID NO: 6 (AGUGAU) and SEQ ID NO: 7 (AGUGUA).

Generally, mutations are envisioned by one of skill in the art at any position in the nucleotide motif sequence that do not affect the function of the nucleotide motif, for example the nuclear targeting function. Accordingly, in certain examples, the localization element comprises SEQ ID NO: 8 (AGNGUN), where N is any nucleotide.

Small RNAs: Small Inhibitory RNAS and microRNAs

Small RNA molecules are common and effective modulators of gene expression in a variety of eukaryotic cells. Small ribonucleic acid (RNA) molecules are short RNA sequences (e.g., 15 to 30 nucleotides in size, but generally 21-24 nucleotides in size) that are produced by nearly all eukaryotes (e.g., fungi, plants, and animals). However, rather than encoding a protein, small RNAs function to reduce the mRNA abundance or protein abundance of the gene which is the “target.” In certain instances small RNAs can also result in target gene regulation by affecting chromatin structure.

There are a number of different types of small RNAs, including, but not limited to, short interfering (si) RNAs (see, for example, Elbashir, S., Lendeckel, W. and Tuschl, T. (2001). RNA interference is mediated by 21- and 22-nucleotide RNAs. Genes Dev. 15, 188-200), piRNAs, rasRNAs, small temporal (st) RNAs (Pasquinelli, A., Reinhart, B., Slack, F., Martindale, M., Kuroda, M., Mailer, B., Hayward, D., Ball, E., Degnan, B., Muller, P. et al. (2000). Conservation of the sequence and temporal expression of let-7 heterochronic RNA. Nature 408, 86-89), heterochromatic siRNAs (Reinhart, B. and Bartel, D. (2002). Small RNAs correspond to centromere heterochromatic repeats. Science 297, 1831.), tiny noncoding RNAs (Lee, R. and Ambros, V. (2001). An extensive class of small RNAs in Caenorhabditis elegans. Science 294, 862-864) and microRNAs (miRNAs) (Lau, N., Lim, L., Weinstein, E. and Bartel, D. (2001). An abundant class of tiny RNAs with probable regulatory roles in Caenorhabditis elegans. Science 294, 858-862; Lagos-Quintana, M., Rauhut, R., Lendeckel, W. and Tuschl, T. (2001). Identification of novel genes coding for small expressed RNAs. Science 294, 853-858). Although relatively short in length, 15 to 30 nucleotides, small RNAs typically correspond to a single location in the host genome.

While similar in size, the biogenesis and function of small RNAs can be substantially different. For instance, siRNAs are processed from longer double-stranded RNA molecules and represent both strands of the RNA. In addition, siRNAs are incorporated into a multi-protein complex known as the RNA-induced silencing complex (RISC), where they can act as guides to target and degrade complementary mRNA molecules. In some systems, siRNAs can also trigger transcriptional silencing by guiding nuclear complexes that target either histone modifications or DNA methylation or both.

Alternatively, microRNA molecules originate from distinct genomic loci predicted to encode transcripts that form ‘hairpin’ structures. These small RNAs, which are derived from one strand of the hairpin, guide the RISC (or a similar RNA-protein complex) to specific RNAs, such as mRNAs by forming base-pairing interactions. Like siRNA, miRNAs can induce cleavage and accelerate degradation of the mRNA targets. A second mechanism by which miRNAs affect gene function is to reduce or prevent mRNA translation and thereby limit protein production.

RNA interference refers to the process of sequence-specific post-transcriptional gene silencing in higher eukaryotic cells mediated by short interfering RNAs (siRNAs) (Fire et al., Nature 391:806-811, 1998). RNAi is a sequence-specific post-transcriptional gene silencing mechanism triggered by double-stranded RNA (dsRNA). RNA interference (RNAi) has been used to silence the expression of a target gene. It causes degradation of mRNAs homologous in sequence to the dsRNA. The mediators of the degradation are 21-23-nucleotide small interfering RNAs (siRNAs) generated by cleavage of longer dsRNAs (including hairpin RNAs) by DICER, a ribonuclease III-like protein. Molecules of siRNA typically have 2-3-nucleotide 3′ overhanging ends resembling the RNAse III processing products of long dsRNAs that normally initiate RNAi. When introduced into a cell, they assemble an endonuclease complex (RNA-induced silencing complex), which then guides target mRNA cleavage. As a consequence of degradation of the targeted mRNA, cells with a specific phenotype of the suppression of the corresponding protein product are obtained (e.g., reduction of tumor size, metastasis, angiogenesis, and growth rates).

The small size of siRNAs, compared with traditional antisense molecules, prevents activation of the dsRNA-inducible interferon system present in mammalian cells. This helps avoid the nonspecific phenotypes normally produced by dsRNA larger than 30 base pairs in somatic cells. See, e.g., Elbashir et al., Methods 26:199-213 (2002); McManus and Sharp, Nature Reviews 3:737-747 (2002); Hannon, Nature 418:244-251 (2002); Brummelkamp et al., Science 296:550-553 (2002); Tuschl, Nature Biotechnology 20:446-448 (2002); U.S. Application US2002/0086356 A1; WO 99/32619; WO 01/36646; and WO 01/68836, incorporated by reference in their entireties herein.

The instant invention describes RNA molecules that comprise a first segment comprising a nucleotide motif comprising a localization element, as described herein, and a second segment comprising a region of nucleotides adjacent to the localization element. The RNA molecules can be small RNAs. In preferred embodiments, the RNA is selected from a small interfering RNA (siRNA), a microRNA (miRNA), piRNA, rasiRNA, or any small inhibitory RNA.

As mentioned herein, the miRNA machinery consists of the Dicer ribonuclease and the effector complex known as RISC. RISC contains a member of the PIWI/Argonaute protein family (Hammond, S. M., Boettcher, S., Caudy, A. A., Kobayashi, R. & Hannon, G. J. (2001) Science 293, 1146-1150, Jaronczyk, K., Carmichael, J. B. & Hobman, T. C. (2005) Biochem. J. 387, 561-571). Evidence is emerging that miRNAs may constitute a major mechanism of translational regulation during spermatogenesis in the mouse. Members of the PIWI protein family have been shown to be integral to the completion of spermatogenesis (Deng, W. & Lin, H. (2002) Dev. Cell 2, 819-830; Kuramochi-Miyagawa, S., Kimura, T., Ijiri, T. W., Isobe, T., Asada, N., Fujita, Y., Ikawa, M., Iwai, N., Okabe, M. & Deng, W., et al. (2004) Development), and miRNAs are expressed in male germ cells. In addition, it has been shown in vitro that transition protein 2 (TP2) mRNA, a posttranscriptionally regulated spermiogenic mRNA, is targeted by miR-122a, an miRNA expressed in late germ cells (Yu, Z., Raabe, T. & Hecht, N. B. (2005) Biol. Reprod. 73, 427-433).

Another class of small non-coding RNAs is the repeat-associated small interfering RNAs (rasiRNAs). RasiRNAs were first identified in Drosophila melanogaster. The rasiRNAs are associated with repeated sequences, transposable elements, satellite and microsatellite DNA, and Suppressor of Stellate repeats, suggesting that small RNAs may participate in defining chromatin structure (Aravin, et al., Dev. Cell, 2003, 5, 337-350). Unlike siRNAs and miRNAs, rasiRNAs function through the Piwi, rather than the Ago, Argonaute protein subfamily.

Tiny non-coding RNA (tncRNA), one class of small non-coding RNAs (Ambros et al., Curr. Biol., 2003, 13, 807-818) produce transcripts similar in length (20-21 nucleotides) to miRNAs, and are also thought to be developmentally regulated but, unlike miRNAs, tncRNAs are reportedly not processed from short hairpin precursors and are not phylogenetically conserved. Although none of these tncRNAs are reported to originate from miRNA hairpin precursors, some are predicted to form potential foldback structures reminiscent of pre-miRNAs; these putative tncRNA precursor structures deviate significantly from those of pre-miRNAs in key characteristics, i.e., they exhibit excessive numbers of bulged nucleotides in the stem or have fewer than 16 base pairs involving the small RNA (Ambros et al., Curr. Biol., 2003, 13, 807-818).

Adjacent is meant to refer to the position of the localization element in relation to the second segment comprising a region of nucleotides. In preferred embodiments, adjacent refers to the position of the localization element as 3′ to the second segment comprising a region of nucleotides.

The first segment and the second segment both comprise a double-stranded region of nucleotides, and together comprise a region of about 15-80 nucleotides in length, preferably about 21-23 nucleotides preferred length. Together, the first and second segments form an RNA duplex. It is preferred in certain embodiments, that the first segment and the second segment comprising a double-stranded region of nucleotides are annealed such that there is a 2-nucleotide 3′ overhang. In certain embodiments, a single-stranded hairpin covalently links the sense and antisense RNA strands that form an RNA duplex.

Together, the second segment double-stranded region of nucleotides adjacent to the first segment double stranded region of nucleotides provides specificity of the RNA to a target gene. Accordingly, the RNA molecule is targeted to any stretch of approximately 20-25 contiguous nucleotides in a target mRNA sequence.

In certain examples, the localization element of the RNA molecule or the siRNA or miRNA molecule is selected from SEQ ID NO: 1 (AGUGUU), SEQ ID NO: 2 (UGUGUU), SEQ ID NO: 3 (ACUGUU), SEQ ID NO: 4 (AGAGUU), SEQ ID NO: 5 (AGUCUU), SEQ ID NO: 6 (AGUGAU) and SEQ ID NO: 7 (AGUGUA). In other examples, the localization element of the RNA molecule or the siRNA or miRNA molecule comprises SEQ ID NO: 8 (AGNGUN), where N is any nucleotide. As above, one of skill in the art can envision any number of mutations of the nucleotide motifs.

According to the invention, the localization element of the RNA molecule or the siRNA or miRNA molecule is capable of inhibiting the expression of a target gene in a cell. The target gene may be one or more of an endogenous cellular gene, an exogenous gene or a viral gene. The target gene can be of mammalian origin. The target gene can be of human origin.

In certain examples, it is possible that the target gene is expressed in a phenotypically normal cell or is expressed in a diseased cell. A target cell may be considered to be any cell that has increased activity of a gene of interest. A phenotypically normal cell refers to a cell that is functioning normally. For example, a phenotypically normal cell may not display a diseased phenotype, but may produced increased levels of a gene product, for example a hormone or a signaling protein, and thus gene silencing may still be desired. A diseased cell may be any type of cell, for example a tumor cell.

In effecting gene silencing, the small RNA molecule or the siRNA or miRNA molecule may inhibit expression of the target gene by at least 40%, 50%, 60%, 70%, 75%, 80%, 85%, 90%, 95%, or more.

The RNA molecule or the siRNA or miRNA molecule in certain examples directs nuclear localization of the RNA, enhances nuclear localization of the RNA, or increases the nuclear accumulation of the RNA, or any combination of the above.

International PCT Publication No. WO 00/01846, describes certain methods for identifying specific genes responsible for conferring a particular phenotype in a cell using specific dsRNA molecules. International PCT Publication No. WO 01/29058 describes the identification of specific genes involved in dsRNA-mediated RNAi. International PCT Publication No. WO 99/07409, describes specific compositions consisting of particular dsRNA molecules combined with certain anti-viral agents. International PCT Publication No. 99/53050 describes certain methods for decreasing the phenotypic expression of a nucleic acid in plant cells using certain dsRNAs. International PCT Publication No. WO 01/49844 describes specific DNA constructs for use in facilitating gene silencing in targeted organisms.

International PCT Publications Nos. WO 02/055692, WO02/055693, and EP 1144623 describe certain methods for inhibiting gene expression using RNAi. International PCT Publications Nos. WO 99/49029 and WO01/70949, and AU 4037501 describe certain vectors expressing siRNA molecules. U.S. Pat. No. 6,506,559, describes certain methods for inhibiting gene expression in vitro using certain siRNA constructs that mediate RNAi.

Selecting Target Sequences

The siRNA or miRNA of the invention can be targeted to any stretch of approximately 19 25 contiguous nucleotides in any of the target mRNA sequences. Techniques for selecting target sequences for siRNA are known in the art and are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany. Thus, the sense strand of the small RNAs (siRNA or miRNA) of the instant invention comprises a nucleotide sequence identical to any contiguous stretch of about 19 to about 25 nucleotides in the target mRNA.

Suitable siRNA sequences can be identified using any means known in the art. Typically, the methods described in Elbashir et al., Nature, 411:494-498 (2001) and Elbashir et al., EMBO J., 20:6877-6888 (2001) are combined with rational design rules set forth in Reynolds et al., Nature Biotech., 22(3):326-330 (2004). A number of tools are available on the World Wide Web on commercially available sites.

Nucleotides 2 to 7 of microRNAs (miRNAs), known as “seed” sequences, are considered the most critical for selecting targets. Within a given species, highly related miRNAs sharing a common seed sequence are grouped into miRNA families, are predicted to have overlapping targets, and are considered to be largely redundant (M. A. Valencia-Sanchez, J. Liu, G. J. Hannon, R. Parker, Genes Dev. 20, 515 (2006); T. Du, P. D. Zamore, Development 132, 4645 (2005); J. Brennecke, A. Stark, R B. Russell, S. M. Cohen, PLoS Biol. 3, e85 (2005); E. C. Lai, Genome Biol. 5, 115 (2004); B. P. Lewis, C. B. Burge, D. P. Bartel, Cell 120, 15(2005). Nevertheless, loss of function of miRNA family members with divergent 3′ end sequences results in overlapping but distinct phenotypes in Caenorhabditis elegans and in Drosophila (D. Leaman et al., Cell 121, 1097 (2005). A. L. Abbott et al., Dev. Cell 9, 403 (2005).). These distinct phenotypes often do not appear to be due to differences in miRNA expression patterns, which raises the possibility that distinct sequences within miRNA family members confer upon them characteristic functional properties.

Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon.

Generally, the nucleotide sequence 3′ of the AUG start codon of a transcript from the target gene of interest is scanned for dinucleotide sequences (e.g., AA, NA, CC, GG, or UU, wherein N.dbd.C, G, or U) (see, e.g., Elbashir et al., EMBO J., 20:6877-6888 (2001)). The nucleotides immediately 3′ to the dinucleotide sequences are identified as potential siRNA target sequences. Typically, the 19, 21, 23, 25, 27, 29, 31, 33, 35, or more nucleotides immediately 3′ to the dinucleotide sequences are identified as potential siRNA target sites. In some embodiments, the dinucleotide sequence is an AA or NA sequence and the 19 nucleotides immediately 3′ to the AA or NA dinucleotide are identified as a potential siRNA target site. siRNA target sites are usually spaced at different positions along the length of the target gene. To further enhance silencing efficiency of the siRNA sequences, potential siRNA target sites may be analyzed to identify sites that do not contain regions of homology to other coding sequences, e.g., in the target cell or organism. For example, a suitable siRNA target site of about 21 base pairs typically will not have more than 16-17 contiguous base pairs of homology to coding sequences in the target cell or organism. If the siRNA sequences are to be expressed from an RNA Pol III promoter, siRNA target sequences lacking more than 4 contiguous A's or T's are selected.

Once a potential siRNA sequence has been identified, the sequence can be analyzed using a variety of criteria known in the art. For example, to enhance their silencing efficiency, the siRNA sequences may be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. siRNA design tools that incorporate algorithms that assign suitable values of each of these features and are useful for selection of siRNA can be found at, e.g., http://box094.ust.hk/RNAi/siRNA. One of skill in the art will appreciate that sequences with one or more of the foregoing characteristics may be selected for further analysis and testing as potential siRNA sequences.

Small RNA (siRNA or miRNA) molecules can be provided in several forms including, e.g., as one or more isolated siRNA or miRNA duplexes. The miRNA or siRNA sequences may have overhangs (e.g., 3′ or 5′ overhangs as described in Elbashir et al., Genes Dev., 15:188 (2001) or Nykanen et al., Cell, 107:309 (2001), or may lack overhangs (i.e., have blunt ends).

Producing siRNAs or miRNAs

The siRNA of the invention can be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference.

US Published Application 20070044164, incorporated by reference in its entirety herein, describes methods for producing miRNAs, in particular, recombinant vectors for inducible and/or tissue specific expression of double-stranded RNA molecules that interfere with the expression of a target gene.

The small RNAs of the invention may be chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA or miRNA can also be expressed from recombinant circular or linear DNA plasmids using any suitable promoter. Suitable promoters for expressing siRNA or miRNA include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA or miRNA in a particular tissue or in a particular intracellular environment.

The siRNA expressed from recombinant plasmids can either be isolated from cultured cell expression systems by standard techniques, or can be expressed intracellularly at or near the area of neovascularization in vivo.

siRNA or miRNA, piRNA, rasiRNA or any small RNA of the invention can be expressed from a recombinant plasmid either as two separate, complementary RNA molecules, or as a single RNA molecule with two complementary regions.

Selection of plasmids suitable for expressing siRNA, miRNA or any small RNA of the invention, methods for inserting nucleic acid sequences for expressing the siRNA into the plasmid, and methods of delivering the recombinant plasmid to the cells of interest are within the skill in the art. See, for example Tuschl, T. (2002), Nat. Biotechnol, 20: 446 448; Brummelkamp T R et al. (2002), Science 296: 550 553; Miyagishi M et al. (2002), Nat. Biotechnol. 20: 497 500; Paddison P J et al. (2002), Genes Dev. 16: 948 958; Lee N S et al. (2002), Nat. Biotechnol. 20: 500 505; and Paul C P et al. (2002), Nat. Biotechnol. 20: 505 508, the entire disclosures of which are herein incorporated by reference.

As used herein, “in operable connection with a polyT termination sequence” means that the nucleic acid sequences encoding the sense or antisense strands are immediately adjacent to the polyT termination signal in the 5′ direction. During transcription of the sense or antisense sequences from the plasmid, the polyT termination signals act to terminate transcription.

As used herein, “under the control” of a promoter means that the nucleic acid sequences encoding the sense or antisense strands are located 3′ of the promoter, so that the promoter can initiate transcription of the sense or antisense coding sequences.

The siRNA or miRNA, or any small RNA of the invention can also be expressed from recombinant viral vectors intracellularly at or near the area of neovascularization in vivo. The recombinant viral vectors of the invention comprise sequences encoding the siRNA or miRNA or any small RNA of the invention and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA or miRNA in a particular tissue or in a particular intracellular environment. The use of recombinant viral vectors to deliver siRNA or miRNA of the invention to cells in vivo is discussed in more detail below.

SiRNA or miRNA or any small RNA of the invention can be expressed from a recombinant viral vector either as two separate, complementary RNA molecules, or as a single RNA molecule with two complementary regions.

Any viral vector capable of accepting the coding sequences for the siRNA or miRNA molecule(s) to be expressed can be used, for example vectors derived from adenovirus (AV); adeno-associated virus (AAV); retroviruses (e.g. lentiviruses (LV), Rhabdoviruses, murine leukemia virus); herpes virus, and the like. The tropism of the viral vectors can also be modified by pseudotyping the vectors with envelope proteins or other surface antigens from other viruses.

Selection of recombinant viral vectors suitable for use in the invention, methods for inserting nucleic acid sequences for expressing the siRNA or miRNA into the vector, and methods of delivering the viral vector to the cells of interest are within the skill in the art. See, for example, Domburg R (1995), Gene Therap. 2: 301 310; Eglitis M A (1988), Biotechniques 6: 608 614; Miller A D (1990), Hum Gene Therap. 1: 5 14; and Anderson W F (1998), Nature 392: 25 30, the entire disclosures of which are herein incorporated by reference.

Preferred viral vectors are those derived from AV and AAV. In a particularly preferred embodiment, the siRNA or miRNA of the invention is expressed as two separate, complementary single-stranded RNA molecules from a recombinant AAV vector comprising, for example, either the U6 or H1 RNA promoters, or the cytomegalovirus (CMV) promoter.

A suitable AV vector for expressing the siRNA or miRNA of the invention, a method for constructing the recombinant AV vector, and a method for delivering the vector into target cells, are described in Xia H et al. (2002), Nat. Biotech. 20: 1006 1010.

Suitable AAV vectors for expressing the siRNA or miRNA of the invention, methods for constructing the recombinant AV vector, and methods for delivering the vectors into target cells are described in Samulski R et al. (1987), J. Virol. 61: 3096 3101; Fisher K J et al. (1996), J. Virol., 70: 520 532; Samulski Ret al. (1989), J. Virol. 63: 3822 3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

Evaluation of siRNA or miRNA Effect

The ability of an siRNA an miRNA, piRNA, rasiRNA or any small RNA molecule containing a given target sequence to cause RNAi-mediated degradation of the target mRNA can be evaluated using standard techniques for measuring the levels of RNA or protein in cells. For example, siRNA of the invention can be delivered to cultured cells, and the levels of target mRNA can be measured by Northern blot or dot blotting techniques, or by quantitative RT-PCR.

Small RNA-mediated degradation of the target mRNA can be detected by measuring levels of the target mRNA or protein in the cells of a subject, using standard techniques for isolating and quantifying mRNA or protein as described above

Modified RNA

The invention also includes modified small RNAs. For example, a modified siRNA, miRNA, piRNA, rasiRNA or any small RNA with a modification.

Exemplary modifications include, but are not limited to, modifications that increase the stability of the RNA, for example chemical modifications to the RNA backbone, or modifications that link the RNA to molecules that target it to certain cells, such as antibodies or aptamers. Still other modifications include those that increase cellular permeability, for example a cholesterol linkage.

In certain examples, the modification is a chemical modification. US Publication No. 20070135372, incorporated by reference in its entirety herein, describes chemically modified siRNA molecules and methods of using such siRNA molecules to silence target gene expression. Accordingly, minimal chemical modifications, such as 2′-O-methyl (2′OMe) modifications, at selective positions within one or both strands of the siRNA duplex are sufficient to reduce or completely abrogate the immunostimulatory activity of siRNA. In certain instances, by restricting chemical modification to the non-targeting sense strand of the siRNA duplex, the immunostimulatory activity of siRNA can be abolished while retaining full RNAi activity. Alternatively, minimal chemical modifications, such as 2′OMe modifications, at selective positions within the sense and antisense strands of the siRNA duplex are sufficient to decrease the immunostimulatory properties of siRNA while retaining RNAi activity. Typically, the modified siRNA comprises from about 1% to about 100% (e.g., about 1%, 5%, 10%, 15%, 20%, 25%, 30%, 35%, 40%, 45%, 50%, 55%, 60%, 65%, 70%, 75%, 80%, 85%, 90%, 95%, or 100%) modified nucleotides in the double-stranded region of the siRNA duplex. The modified siRNA can comprise modified nucleotides in one strand (i.e., sense or antisense) or both strands of the double-stranded region of the siRNA. The modified siRNA in some embodiments may comprise a carrier system, e.g., to deliver the modified siRNA into a cell of a mammal. Non-limiting examples of carrier systems suitable for use in the present invention include nucleic acid-lipid particles, liposomes, micelles, virosomes, nucleic acid complexes, and mixtures thereof. In certain instances, the modified siRNA, miRNA or any small RNA molecule is complexed with a lipid such as a cationic lipid to form a lipoplex. In certain other instances, the modified siRNA, miRNA or any small RNA molecule is complexed with a polymer such as a cationic polymer (e.g., polyethylenimine (PEI) to form a polyplex. The modified siRNA, miRNA or any small RNA molecule may also be complexed with cyclodextrin or a polymer thereof. Preferably, the modified siRNA molecule is encapsulated in a nucleic acid-lipid particle.

For example, studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity (Elbashir et al., 2001, EMBO J., 20, 6877). In addition, Elbashir et al, supra, also report that substitution of siRNA with 2′-O-methyl nucleotides completely abolishes RNAi activity.

Target Sequences

The RNA molecules described herein, for example in exemplary embodiments the miRNA and the siRNA, or a piRNA, rasiRNA or any small RNA molecule as described herein, can be used to downregulate or silence the translation (i.e., expression) of a gene of interest. Virtually any gene of interest in the hands of the skilled artisan can be a target of the RNA molecules of the invention. In certain embodiments, genes of interest include, but are not limited to, genes associated with viral infection and survival, genes associated with metabolic diseases and disorders (e.g., liver diseases and disorders), genes associated with tumorigenesis and cell transformation, angiogenic genes, immunomodulator genes such as those associated with inflammatory and autoimmune responses, ligand receptor genes, and genes associated with neurodegenerative disorders. Genes of interest are not limited to those expressed in diseased cells, but can be genes expressed in any cell. Accordingly, any cell harboring a gene with increased expression is a potential target of silencing using the method of the instant invention.

### Methods

Included in the invention are methods for inhibiting the expression of a target gene in a cell comprising introducing into the cell an effective amount of an RNA molecule as described herein or the siRNA or miRNA molecule as described herein.

Methods of the invention include introducing an RNA molecule that silences expression of a target sequence into a cell comprising contacting the cell with an RNA molecule as described herein or a siRNA or miRNA molecule as described herein.

Small non-coding RNA-mediated regulation of gene expression is an attractive approach to the treatment of diseases as well as infection by pathogens such as bacteria, viruses and prions and other disorders associated with RNA expression or processing.

The invention teaches methods for preventing or treating a disease or disorder, comprising administering to a subject in need thereof a pharmaceutical composition comprising a therapeutically effective amount of an RNA molecule or an siRNA or miRNA molecule as described herein, thereby preventing or treating a disease or disorder.

As discussed above, virtually any gene of interest in the hands of the skilled artisan can be a target of the RNA molecules of the invention, thus any gene involved or believed to be involved in a disease or disorder can be targeted in the methods of the invention. Accordingly, a beneficial therapeutic effect of treating the disease or disorder comprises silencing of at least one gene.

The invention includes methods of making an RNA molecule with enhanced nuclear localization and with specificity to a target gene. The method comprises obtaining a first segment comprising a nucleotide motif comprising a localization element, and a second segment comprising a region nucleotides that provides specificity to a target gene, wherein the first segment is attached to the 3′ terminus of the second segment, and thereby generating a RNA molecule with enhanced nuclear localization and specificity to a target gene.

The invention also includes methods of making an RNA molecule with enhanced nuclear import and specificity to a target gene comprising obtaining a first segment comprising a nucleotide motif, and a second segment comprising a region of nucleotides in length that provides specificity to a target gene, wherein the first segment is attached to the 3′ terminus of the second segment, and thereby generating an RNA molecule with enhanced nuclear import and specificity to a target gene.

The invention also includes methods of making an RNA molecule with enhanced nuclear accumulation and specificity to a target gene comprising obtaining a first segment comprising a nucleotide motif comprising a localization element, and a second segment comprising a region of nucleotides that provides specificity to a target gene, wherein the first segment is attached to the 3′ terminus of the second segment, and thereby generating a RNA molecule with enhanced stability and specificity to a target gene.

The nucleotide motif comprising a localization element can also be used to localize a protein of interest to the nucleus. Accordingly, the invention includes methods of making a protein with enhanced nuclear localization, import and accumulation. The methods include obtaining a first segment comprising a nucleotide motif comprising a localization element, and a second segment comprising a region of amino acids encoding a protein of interest. The first segment is attached to the region of amino acids, a protein with enhanced nuclear localization, import and accumulation is generated. In certain embodiments, the first segment is attached to the second segment with a linker, for example a histidine linker.

RNA Silencing

The RNA molecules of the invention as described herein can silence the expression of the target sequence in a number of different ways. For example, in one embodiment, the silencing is transcriptional silencing. In another embodiment, gene silencing is a post-transcriptional event.

Gene silencing refers to the suppression or inhibition of expression of the target gene. The term silencing is not necessarily meant to imply reduction of transcription, because gene silencing is believed to operate in at least some cases post-transcriptionally. The degree of gene silencing can be complete so as to abolish production of the encoded gene product. As such, complete gene silencing can produce a null phenotype. The degree of gene silencing can be partial. With partial gene silencing, some degree of expression of the gene product remains. Partial gene silencing can produce an intermediate phenotype.

The mRNA molecule, for example inhibitory mRNA molecules including but not limited to siRNAs, miRNAs, piRNAs, rasiRNAs, regulate gene-silencing post-transcriptionally. For example, the mRNA molecule can regulate any aspect of mRNA biogenesis or trafficking, including mRNA splicing, mRNA 3′ end formation, mRNA degradation, or mRNA transport. The above-mentioned methods of silencing are not meant to be limiting and other means of silencing by the RNA molecules are possible. Moreover, it is possible that a localization motif has a translational silencing effect on one small RNA, but a transcriptional silencing effect on another, for example the siRNA and miRNA pathways may be different in that the one leads to mRNA degradation and the other leads to translational repression.

There is a connection between RNAi and chromatin modification that has been investigated in fission yeast. Studies have shown that small RNAs complementary to centromeric repeats and proteins of the RNAi pathway [Dicer, RNA-dependent RNA polymerase (Rdp) and Ago] are required for histone H3K9 methylation and centromere function (Volpe, T., Schramke, V., Hamilton, G. L., White, S. A., Teng, G., Martienssen, R. A. and Allshire, R. C. (2003). RNA interference is required for normal centromere function in fission yeast. Chromosome Research 11, 137-146; Hall, I., Noma, K.-i. and Grewal, S. (2003). RNA interference machinery regulates chromosome dynamics during mitosis and meiosis in fission yeast. Proc. Natl. Acad. Sci. USA 100, 193-198). Similarly, a copy of the centromeric repeat at the mating type locus in S. pombe is also a target of RNAi-mediated heterochromatin formation (Hall, I., Shankaranarayana, G., Noma, Ayoub, N., Coehn, A. and Grewal, S. (2002). Establishment and maintenance of a heterochromatic domain. Science 297, 2232-2237). The centromeric siRNAs, termed ‘heterochromatic siRNAs’ to indicate their involvement in epigenetic modifications, are derived from overlapping transcripts of centromere outer repeats (Reinhart, B. and Bartel, D. (2002). Small RNAs correspond to centromere heterochromatic repeats. Science 297, 1831). RNAi-dependent chromatin modifications can also target long terminal repeats of retrotransposons in S. pombe, thereby repressing adjacent meiotically induced genes in vegetative cells (Schramke, V. and Allshire, R. (2003). Hairpin RNAs and retrotransposons LTRs effect RNAi and chromatin-based gene silencing. Science 301, 1069-1074 [Epub 2003 Jul. 17]). RNAi-induced heterochromatin formation thus appears to be a general means for regulating gene expression in S. pombe.

Methods of Delivery of RNA

The small RNAs as described herein can be chemically synthesized. In other certain embodiment, as described above, the RNA molecule is in an expression vector. A variety of different vectors are known in the art, including but not limited to a plasmid vector and a viral vector. Such vectors generally have convenient restriction sites located near the promoter sequence to provide for the insertion of nucleic acid sequences. Transcription cassettes can be prepared comprising a transcription initiation region, the target gene or fragment thereof, and a transcriptional termination region. The transcription cassettes can be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

In the methods of the invention as described herein, for example in preventing or treating a disease or disorder, the RNA molecule may attenuate the expression of a target gene within a cell ex vivo or attenuate the expression of a target gene within a cell in vivo.

The RNA molecule can be, but is not limited to, systemic administration. Systemic delivery or administration refers to delivery that leads to a broad biodistribution of a compound such as an siRNA within an organism. Some techniques of administration can lead to the systemic delivery of certain compounds, but not others. Systemic delivery means that a useful, preferably therapeutic, amount of a compound is exposed to most parts of the body. To obtain broad biodistribution generally requires a blood lifetime such that the compound is not rapidly degraded or cleared (such as by first pass organs (liver, lung, etc.) or by rapid, nonspecific cell binding) before reaching a disease site distal to the site of administration. Systemic delivery of nucleic acid-lipid particles can be by any means known in the art including, for example, intravenous, subcutaneous, and intraperitoneal.

Delivery can also be local. By local delivery is meant delivery of a compound such as an siRNA directly to a target site within an organism. For example, a compound can be locally delivered by direct injection into a disease site such as a tumor or other target site such as a site of inflammation or a target organ such as the liver, heart, pancreas, kidney, and the like.

Methods for the delivery of nucleic acid molecules are described in Akhtar et al., (Trends Cell Bio. 2, 139, 1992). WO 94/02595 describes general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport systems, for example, through the use of conjugates and biodegradable polymers. More detailed descriptions of nucleic acid delivery and administration are provided for example in WO93/23569, WO99/05094, and WO99/04819.

In certain embodiments, the modified siRNA is delivered in a carrier system. US Published Application 20070135372 described the use of siRNA in carrier systems.

In one aspect, the present invention provides carrier systems containing the modified siRNA molecules described herein. In some embodiments, the carrier system is a lipid-based carrier system such as a stabilized nucleic acid-lipid particle (e.g., SNALP or SPLP), cationic lipid or liposome nucleic acid complexes (i.e., lipoplexes), a liposome, a micelle, a virosome, or a mixture thereof. In other embodiments, the carrier system is a polymer-based carrier system such as a cationic polymer-nucleic acid complex (i.e., polyplex). In additional embodiments, the carrier system is a cyclodextrin-based carrier system such as a cyclodextrin polymer-nucleic acid complex. In further embodiments, the carrier system is a protein-based carrier system such as a cationic peptide-nucleic acid complex. Preferably, the carrier system is a stabilized nucleic acid-lipid particle such as a SNALP or SPLP. One skilled in the art will appreciate that the modified siRNA molecule of the present invention can also be delivered as naked siRNA.

In certain examples, the carrier system is selected from the group of, but not limited to, a nucleic acid-lipid particle, a liposome, a micelle, a virosome, a nucleic acid complex, and a mixture thereof.

The nucleic acids can be introduced into tissues or host cells by any number of routes, including viral infection, microinjection, or fusion of vesicles. Jet injection may also be used for intramuscular administration, as described by Furth et al. (Anal Biochem 115 205:365-368, 1992). The nucleic acids can be coated onto gold microparticles, and delivered intradermally by a particle bombardment device, or “gene gun” as described in the literature (see, for example, Tang et al. Nature 356:152-154, 1992), where gold microprojectiles are coated with the DNA, then bombarded into skin cells.

The siRNA can be directly introduced into the cell (i.e., intracellularly); or introduced extracellularly into a cavity, interstitial space, into the circulation of an organism, introduced orally, etc. Methods for oral introduction include direct mixing of RNA with the food of the organism. Physical methods of introducing nucleic acids include injection directly into the cell or extracellular injection into the organism of an RNA solution. The agent can be introduced in an amount that allows delivery of at least one functional copy per cell. Higher doses (e.g., at least 5, 10, 100, 500 or 1000 or more copies per cell) of the agent may yield more effective inhibition; lower doses may also be useful for specific applications.

Other methods known in the art for introducing nucleic acids to cells can be used, including for example lipid-mediated carrier transport, electroporation of cell membranes, chemical-mediated transport such as calcium phosphate, and the like. Thus the RNA may be introduced along with components that perform one or more of the following activities: enhance RNA uptake by the cell, promote annealing of the duplex strands, stabilize the annealed strands, or otherwise increase inhibition of the target gene.

The expression of the RNA can be constitutive or regulatable. For example, the nucleic acid encoding the RNAi may be located on the vector where it is operatively linked to an appropriate expression control sequence e.g., the tetracyline repressor as described for example in International Patent Publication No. WO04/065613.

To adapt RNAi for the study of gene function in animals, genetic engineering can be used to create mouse embryonic stem cells in which RNAi is targeted to a particular gene (Carmell et al., Nat Struct Biol. 10(2): 91-92, 2003). This is based on a previous study in which silencing a gene of interest through RNAi was efficiently achieved by engineering a second gene that encoded short hairpin RNA molecules corresponding to the gene of interest (Carmell et al., 2003). The stem cells were injected into mouse embryos, and chimeric animals were born. Subsequent mating of these chimeric mice produced offspring that contained the genetically engineered RNAi-inducing gene in every cell of their bodies. It was observed from examination of the tissues from transgenic mice, that the expression of the gene of interest was significantly reduced throughout the organism (e.g. liver, heart, spleen). Such a reduction in gene expression is called a “gene knockdown” to distinguish it from traditional methods that involve “gene knockouts” or the complete deletion of a DNA segment from a chromosome. One advantage of this RNAi-based gene knockdown strategy, is that the strategy can be modified to silence the expression of genes in specific tissues, and it can be designed to be switched on and off at any time during the development or adulthood of the animal.

The cells may be transfected or otherwise genetically modified ex vivo. The cells are isolated from a mammal (preferably a human), nucleic acid introduced (i.e., transduced or transfected in vitro) with a vector for expressing an RNAi and then administered to a mammalian recipient for delivery of the therapeutic agent in situ. The mammalian recipient may be a human and the cells to be modified are autologous cells, i.e., the cells are isolated from the mammalian recipient. According to another embodiment of the present invention, the cells are transfected or transduced or otherwise genetically modified in vivo. The cells from the mammalian recipient are transduced or transfected in vivo with a vector containing exogenous nucleic acid material for expressing an RNAi and the therapeutic agent is delivered in situ.

Recently, techniques have been developed to trigger siRNA into a specific target cell (e.g. embryogenic stem cell, hematopoietic stem cell, or neuronal cell) by introducing exogenously produced or intracellularly expressed siRNAs as described for example in WO03/022052 and U.S. Patent Application 2005042646.

Depending on the nature of the RNAi agent, the active agent(s) can be administered to the host using any convenient means capable of resulting in the desired modulation of target gene expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc.

The RNA (the siRNA or miRNA) of the invention can administered to a subject in combination with a pharmaceutical agent that is different from the present siRNA or miRNA. Alternatively, the siRNA of the invention can be administered to a subject in combination with another therapeutic method designed to treat the disease of interest. For example, if the siRNA or miRNA is targeted at treating cancer, then the siRNA of the invention can be administered in combination with other therapeutic methods currently employed for treating cancer or preventing tumor metastasis (e.g., radiation therapy, chemotherapy, and surgery). For treating tumors, the siRNA of the invention is administered to a subject in combination with radiation therapy, or in combination with chemotherapeutic agents such as, but not limited to, cisplatin, carboplatin, cyclophosphamide, 5-fluorouracil, adriamycin, daunorubicin or tamoxifen.

### Monitoring RNA Inhibition

In certain aspects of the invention, it will be advantageous to monitor the effects of RNA inhibition as described herein. The consequences of inhibition can be confirmed by examination of the phenotypical properties of the cell or organism, or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression can be assayed by use of a reporter or drug resistance gene whose protein product is easily assayed. Such assays are routinely performed by one of skill in the art.

### Kits

The invention includes kits. Accordingly, the kits of the invention can comprise the nucleotide motif comprising a localization element as described herein, and instructions for use. Kits can comprise the RNA molecule as described herein, and instructions for use. Kits can comprise the pharmaceutical composition, and instructions for use.

### Examples

It should be appreciated that the invention should not be construed to be limited to the examples that are now described; rather, the invention should be construed to include any and all applications provided herein and all equivalent variations within the skill of the ordinary artisan.

In the Examples described herein, it is shown that despite their small size, specific miRNAs contain additional sequence elements that control their posttranscriptional behavior, including their subcellular localization. In certain exemplary embodiments described herein, it is shown that human miR-29b, in contrast to other studied animal miRNAs, is predominantly localized to the nucleus. Described in the examples is a hexanucleotide motif; however a nucleotide motif comprising any number of nucleotides can be envisioned according to the instant invention. The distinctive hexanucleotide terminal motif of miR-29b acts as a transferable nuclear localization element that directs nuclear enrichment of miRNAs or small interfering RNAs to which it is attached. The results described herein indicate that miRNAs sharing common 5′ sequences, considered to be largely redundant, might have distinct functions because of the influence of cis-acting regulatory motifs.

### Materials and Methods

The following materials and methods were employed in the below examples.

### Cell Synchronization and Mitotic Arrest

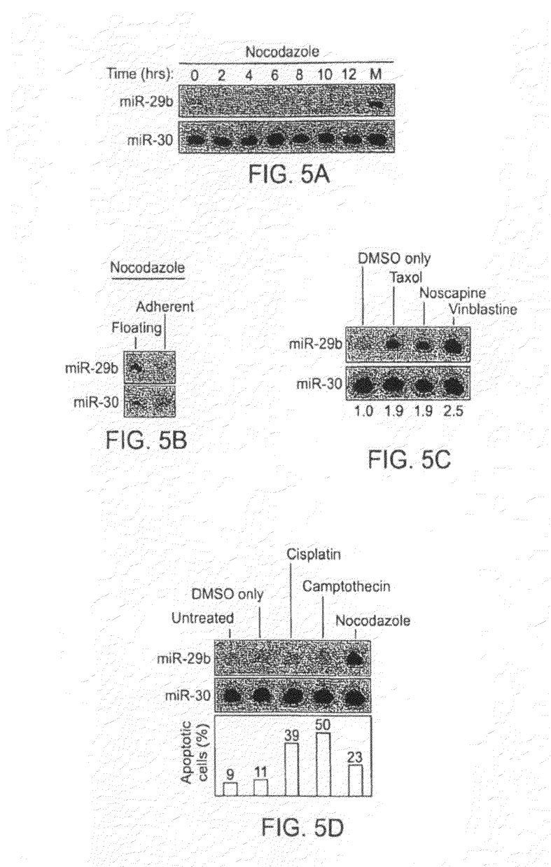

HeLa cells and NIH3T3 cells were grown in Dulbecco's modified Eagle's medium supplemented with 10% fetal bovine serum. Synchronized HeLa cell populations were obtained by double-thymidine block as described (14). Briefly, cells were blocked for 17 hours with 2 mM thymidine, released into regular media for 9 hours, and then blocked again with 2 mM thymidine for 14 hours. Cells were then released into regular media and harvested at various time-points. For all experiments except those shown in FIG. S4C, mitotic cells were obtained by releasing synchronized cells into media containing 100 ng/mL nocodazole. The floating fraction was then harvested 13 hours after release. In the experiment shown in FIG. 5C, synchronized cells were released into media containing DMSO (vehicle), 10 μM taxol, 10 μM vinblastine, or 100 μM noscapine. Again, floating cells were harvested 13 hours later. All microtubule poisons were purchased from Sigma. Cell-cycle profiles were analyzed by propidium iodide (PI) staining in which cells were fixed in 70% ethanol overnight at −20 O C and then stained in 1× PBS containing 50 μg/mL PI and 100 μg/mL RNase A for 1 hour at 4 O C. DNA content was then measured by flow cytometry.

### Induction of Apoptosis

Camptothecin and cisplatin were purchased from Sigma. To induce apoptosis, HeLa cells were treated with 5 μM camptothecin or 20 μM cisplatin for 12 hours. Percentages of apoptotic cells were measured by flow cytometry using Annexin V-PE Apoptosis Detection Kit (BD Pharmingen).

### Northern Blot Analysis

Total RNA was harvested using Trizol (Invitrogen) following the manufacturer's protocol. 10-20 μg of total RNA was separated on denaturing 15% polyacrylamide gels and transferred to GeneScreen Plus membranes (Perkin Elmer Life Sciences). Oligonucleotide probes were 5′ end-labeled with [γ-32P] ATP using T4 polynucleotide kinase (Invitrogen). All probe sequences are listed in Table S4 and Table S5 of this supplement. Hybridizations and washes, including those for miR-29a, -29b, and -29c, were performed using Ultrahyb-Oligo buffer (Ambion) at 42 C according to manufacturer's instructions. Radioactive signals were quantified using a Personal FX phosphoimager (Bio-Rad). Membranes were stripped by incubating in boiling 0.1×SSC, 0.1% SDS for 10 minutes and then re-exposed to phosphor screens to verify removal of probe prior to hybridizing with additional probes. To test the specificity of probes for miR-29 paralogs, 1 μL of 10 nM RNA oligos (synthesized by Integrated DNA Technologies) were separated on polyacrylamide gels and probed as described above.

### Transfection of RNA Duplexes and RNA Decay Rate Measurements

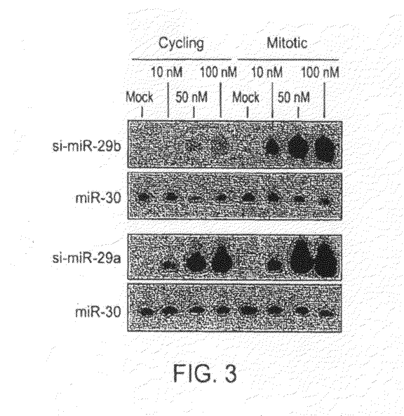

Synthetic RNA duplexes shown in Table 5, below, were obtained from Dharmacon (option A4) and were transfected using Oligofectamine (Invitrogen) according to the manufacturer's protocol. All RNAs were transfected at a final concentration of 25 nM unless otherwise specified. This concentration was chosen because it does not saturate the post-transcriptional mitotic accumulation of miR-29b but is high enough to allow discrimination of RNA derived from exogenous and endogenous sources (see FIG. 3). For FIG. 3, in which the behavior of si-miR-29a and si-miR-29b were examined in cycling cells versus mitotic cells, a single dish of cells was transfected with a given concentration of RNA duplex. After an overnight incubation, cells were split into media with or without nocodazole. This experimental design ensured that differences in transfection efficiency did not account for the observed accumulation of si-miR-29b in mitotic cells.

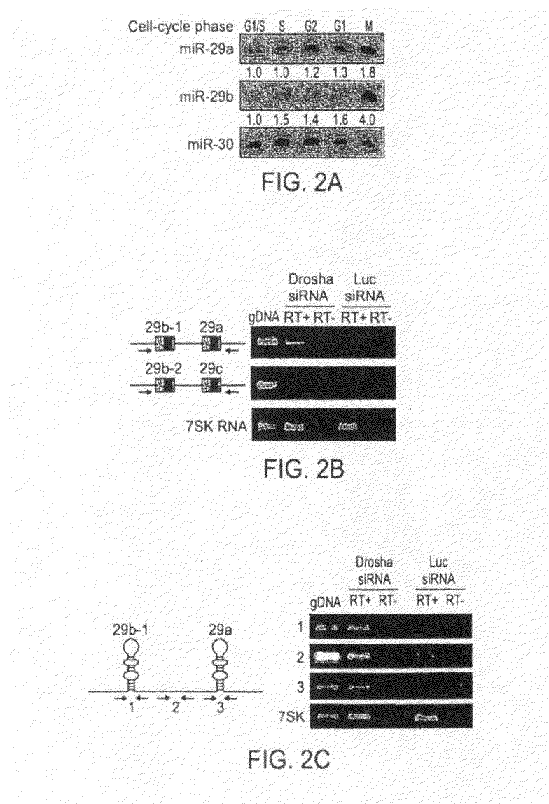

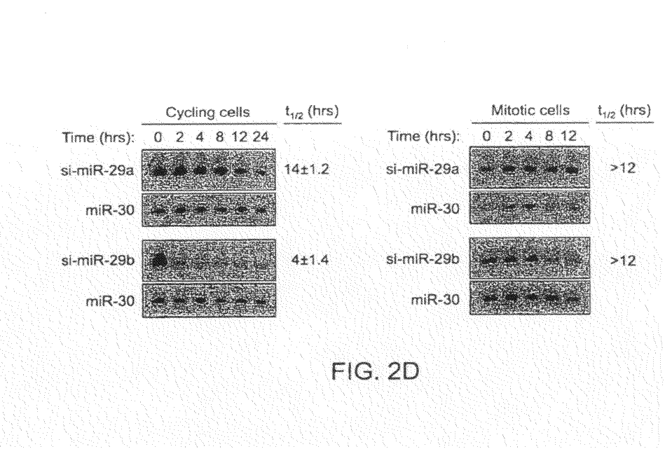

For half-life experiments in cycling cells, oligofectamine/si-miRNA complexes were added to cells for 4 hours and then removed by replacing the media. RNA was harvested at rime-points thereafter and miRNA abundance throughout the time-course was measured by northern blotting. 10 μg of total RNA was loaded for each time-point. Similar results were obtained if overnight transfection was performed. For half-life experiments in mitotic cells, cells were synchronized by double-thymidine block and released into media containing nocodazole. Oligofectamine/si-miRNA complexes were then added and floating cells were collected 12 hours later. Transfection complexes were removed by replacing the media and time-point collection was initiated. Induction of apoptosis by nocodazole precluded prolonging the time-course beyond 12 hours. Half-lives were calculated as described (15). Note that as observed previously for other labile RNAs (15, 16), miR-29b exhibits bi-phasic decay in cycling cells with an initial rapid phase followed by a slower rate of decay at the later time-points (FIG. 2D, compare miR-29b decay in cycling cells from 0-4 hours versus 8-24 hours). All half-lives reported for miR-29b in cycling cells refer to the initial rapid decay phase and were calculated using the first three time-points (0 to 4 hours).

### Drosha Knockdown and RT-PCR

siRNAs directed against Drosha and Luciferase were previously described (Y. Lee et al., Nature 425, 415 (2003); S. M. Elbashir et al., Nature 411, 494 (2001)). Cells were transfected with 100 nM siRNA using Oligofectamine. RNA was harvested with Trizol 72 hours after transfection. Total RNA was further purified using RNeasy columns (Qiagen) followed by DNase I (Invitrogen) digestion. 1 μg RNA was reverse-transcribed using MMLV reverse transcriptase (Invitrogen) with random hexamers prior to amplification. Primer sequences are shown below In Table 1 and Table 2:

TABLE 1
Sequences of primers used in FIG. 2B
(shown 5′ to 3′)

| Amplicon | Forward primer | Reverse primer |
|---|---|---|
| 29b01/29a | TTGTCTTGGGTTTATTGT | CAACGGTCACCAATACATT |
| cluster | AAGAGAGACA | TCCT |
| 29b-2/29c | AGGGCAGAGGCTGGGTCTT | CCATCCATCTTCCAGGAAA |
| cluster | | CC |
| 7SK RNA | GACATCTGTCACCCCATTG | TCTGCAGTCTTGGAAGCTT |
| | ATC | GAC |

TABLE 2
Sequences of primers used in FIG. 2C
(shown 5′ to 3′)

| Amplicon | Forward primer | Reverse primer |
|---|---|---|
| 1 | TTGTCTTGGGTTTATT | ACTTCTTTTGCTGTTGGTAGTGCAG |
| | GTAAGAGAGACA | |
| 2 | AACAGCAGGATGGCTG | AACCCAGGCACAATGGAATGAGTC |
| | TGAGAACC | |
| 3 | ACGACCTTCTGTGACC | CAACGGTCACCAATACATTTCCT |
| | CCTTAGA | |
| 7SK RNA | GACATCTGTCACCCCA | TCTGCAGTCTTGGAAGCTTGAC |
| | TTGATC | |

Analysis of Nuclear and Cytoplasmic miRNA Abundance

Nuclear and cytoplasmic RNA was isolated from untransfected cells (for analysis of endogenous miRNAs) or cells transfected 24 hours previously (for analysis of synthetic RNAs). Cells growing in 10 cm dishes were rinsed twice with ice-cold 1× PBS, harvested in 1 mL ice-cold 1× PBS by scraping, and centrifuged at 1,000 rpm for 10 minutes. Cell pellets were resuspended by gentle pipetting in 200 μL lysis buffer A [10 mM Tris (pH 8.0), 140 mM NaCl, 1.5 mM MgCl2, 0.5% Nonidet P-40], incubated on ice for 5 minutes, and then centrifuged at 1,000×g for 3 minutes at 4 O C. The supernatant, containing the cytoplasmic fraction, was added to 1 mL Trizol for RNA purification. Nuclear pellets underwent two additional washes with lysis buffer A and a final wash with lysis buffer A containing 1% Tween-40 and 0.5% deoxycholic acid. Purified nuclear pellets were then resuspended in 1 mL Trizol. For northern blotting, ⅓ of the total yield of RNA from nuclear and cytoplasmic fractions was loaded to allow comparison of equal cell equivalents.

### Immunoprecipitation

HeLa cells were co-transfected with si-miR-29b duplexes and HA-tagged hAgo1 or hAgo2 expression plasmids (obtained as a gift from T. Tuschl, Rockefeller University) using Lipofectamine 2000 (Invitrogen). 20 hours after transfection, cells were collected by scraping and resuspended in 500 μL lysis buffer [50 mM Tris (pH 7.5), 150 mM NaCl, 2 mM MgCl2, 2 mM CaCl2, 0.5% Nonidet P-40, 1 mM dithiothreitol]. Lysates were cleared by centrifugation at 16,000×g for 10 minutes. 10% of each cleared lysate was added to 1 mL Trizol and saved for assaying the total input. The remaining lysates were mixed with 3 μg anti-HA mouse monoclonal antibody (Covance) and rotated at 4 O C for 1 hour. 50 μL of a 50% slurry of protein G-agarose beads (Roche) was then added and lysates were rotated for an additional 3 hours at 4 C. The beads were then harvested by centrifugation and washed 8 times in lysis buffer. 80% of the beads were resuspended in 1 mL Trizol for RNA isolation and 20% of the beads were resuspended in 20 μL 2× complete Laemmli buffer for protein analysis.

### Plasmid Construction and Transfection

Plasmids that express miR-29b and miR-522 were constructed by first amplifying the pre-miRNA hairpins plus ˜150 by of flanking genomic sequence. The primer sequences are shown in Table 3 below:

TABLE 3
Sequences of primers for plasmid construction
(shown 5′ to 3′)

| Plasmid | Forward primer | Reverse primer |
|---|---|---|
| miR-29b | ATACCGGCTAGCGC | ATACCGCTCGAGGACCTGACTGCCA |
| | ATGCTCTCCCATCAATAA | TTTGTGA |
| miR-522 | AGGCTCGAGTGAAGCAAG | AGGCTCGAGACTCCAGTTTGGGCAG |
| | GAACTGGAGATGG | CAGAC |

Pre-miR-29b-1 was cloned into pcDNA3.1 (Invitrogen) between the NheI and XhoI sites and pre-miR-522 was cloned into pcDNA3.1/V5-His-TOPO using the TA cloning kit (Invitrogen). The miR-29b (m4) mutation was introduced by site-directed mutagenesis using the QuikChange XL Kit (Stratagene). TransIT-HeLaMONSTER (Minus) or FUGENE 6 (Roche) was used for plasmid transfections.

Table 4, below, shows sequences of northern probes used to detect endogenous RNAs (shown 5′ to 3′).

| Target | Probe sequence |
|---|---|
| miR-29a | AACCGATTTCAGATGGTGCTA |
| miR-28b | AACACTGATTTCAAATGGTGCTA |
| miR-30 | GCTGAGAGTGTAGGATGTTTACA |
| miR-21 | TCAACATCAGTCTGATAAGCTA |
| U8 snRNA | GAATTTGCGTGTCATCCTTGCGCAGGGGCCATGCTAA |
| Lysine-tRNA | CTGATGCTCTACCGACTGAGCTATCCGGGC |

Table 5, below, shows sequences of synthetic RNAs and northern probes.

| | Sequence of duplex | |
|---|---|---|
| Synthetic RNA duplex | (top strand 5′ to 3′, bottom strand 3′ to 5′) | Northern probe (5′ to 3′) |
| si-miR-29a | CCGAUUUCAGAUGGUGCUAUU | AACCGATTTCAGATGGTGCTA |
| | UUGGCUAAAGUCUACCACGAU | |
| si-miR-29b | CACUGAUUUCAAAUGGUGCUAUU | AACACTGATTTCAAATGGTGCTA (guide) |
| | UUGUGACUAAAGUUUACCACGAU | AATAGCACCATTTGAAATCAGTG (passenger) |
| si-miR-29c(C10U) | CCGAUUUCAAAUGGUGCUAUU | AACCGATTTCAAATGGTGCTA |
| | UUGGCUAAAGUUUACCACGAU | |
| si-miR-29a(TM^{25b}) | CACUGAUUUCAGAUGGUGCUAUU | AACACTGATTTCAGATGGTGCTA |
| | UUGUGACUAAAGUCUACCACGAU | |
| si-Luc | CGAUACGCGGAAUACUUCGAUU | AACGTACGCGGAATACTTCGA |
| | UUGCAUGCGCCUUAUGAAGCU | |
| si-Luc(TM^{25b}) | CACUUACGCGGAAUACUUCGAUU | AACACTTACGCGGAATACTTCGA |
| | UUGUGAAUGCGCCUUAUGAAGCU | |
| si-test(TM^{25b}) | CACUACGGCGCCUGGGUAAGAUU | AACACTACGGCGCCTGGGTAAGA |
| | UUGUGAUGCCGCGGACCCAUUCU | |
| si-miR-29b(22 nt) | CACUGAUUCAAAUGGUGCUAUU | AACACTGATTCAAATGGTGCTA |
| | UUGUGACUAAGUUUACCACGAU | |
| si-miR-29b(21 nt) | CACUGAUUCAAUGGUGCUAUU | AACACTGATTCAATGGTGCTA |
| | UUGUGACUAAGUUACCACGAU | |
| si-miR-29b(TM⁻¹) | ACACUAUUUCAAAUGGUGCUAUC | GAACACTATTTCAAATGGTGCTA |
| | CUUGUGAUAAAGUUUACCACGAU | |
| si-miR-29b(TM⁻³) | AACACUUUUCAAAUGGUGCUAUC | GAAACACTTTTCAAATGGTGCTA |
| | CUUUGUGAAAAGUUUACCACGAU | |
| si-miR-29b(m1) | CACAGAUUUCAAAUGGUGCUAUU | AACACAGATTTCAAATGGTGCTA |
| | UUGUGUCUAAAGUUUACCACGAU | |
| si-miR-29b(m2) | CAGUGAUUUCAAAUGGUGCUAUU | AACAGTGATTTCAAATGGTGCTA |
| | UUGUCACUAAAGUUUACCACGAU | |
| si-miR-29b(m3) | CUCUGAUUUCAAAUGGUGCUAUU | AACTCTGATTTCAAATGGTGCTA |
| | UUGAGACUAAAGUUUACCACGAU | |
| si-miR-29b(m4) | GACUGAUUUCAAAUGGUGCUAUU | AAGACTGATTTCAAATGGTGCTA |
| | UUCUGACUAAAGUUUACCACGAU | |
| si-miR-29b(m5) | CACUGAUUUCAAAUGGUGCUAUU | ATCACTGATTTCAAATGGTGCTA |
| | UAGUGACUAAAGUUUACCACGAU | |
| si-miR-29b(m6) | CACUGAUUUCAAAUGGUGCUAUU | TACACTGATTTCAAATGGTGCTA |
| | AUGUGACUAAAGUUUACCACGAU | |

Fluorescent in situ Hybridization and Confocal Microscopy

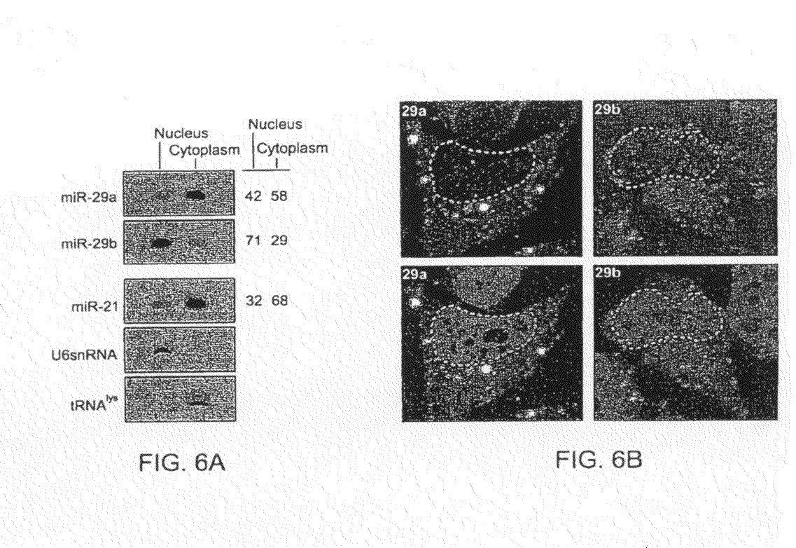

Locked-nucleic acid (LNA) oligonucleotide probes were purchased from Exiqon and end-labeled using the DIG Oligonucleotide Tailing Kit (Roche). Labeled probes were purified using MicroSpin G-25 columns (Amersham). HeLa cells were plated on coverslips a day prior to transfection with si-miR-29a and si-miR-29b duplexes. 24 hours after transfection, cells were washed with 1× PBS, fixed in 4% paraformaldehyde in 1× PBS for 30 minutes, washed again with 1× PBS, and then permeabilized in 70% ethanol at 4 C overnight. The following day, coverslips were rehydrated in 2×SSC-50% formamide for 5 minutes before pre-hybridization at 50 C for 2 hours in hybridization buffer [2×SSC, 50% formamide, 10% dextran sulfate, 2 mM ribonucleoside vanadyl complex (New England Biolabs), 40 μg E. Coli tRNA (Sigma), 0.02% RNase-free BSA (Roche)]. Pre-hybridization buffer was then removed and replaced with hybridization buffer containing 50 nM labeled LNA probe. Hybridization was carried out at 50 C overnight in a humidified chamber. The coverslips were then twice washed in 2×SSC-50% formamide for 30 minutes at 37 O C, blocked in 0.25% BSA-4×SSC for 60 minutes, and incubated with a 1:250 dilution of mouse monoclonal anti-digoxigenin antibody (Roche) in 0.25% BSA-4×SSC for 60 minutes. Coverslips were then washed three times with 4×SSC for 10 minutes each. The signals were further amplified using the Tyramide Signal Amplification Kit #5 (Invitrogen) according to the manufacturer's instructions. After amplification, the coverslips were stained with DAPI and mounted with ProLong antifade reagent (Invitrogen). Cells were imaged at the Johns Hopkins University School of Medicine Microscopy Facility using a Zeiss LSM 510 META confocal microscope. Staining patterns were very uniform on each slide with at least 80% of cells in several assayed high-power fields exhibiting the localization patterns shown in FIG. 6B. Slides lacking LNA probe or primary antibody, included in all experiments, ruled out significant autofluorescence or other non-specific signals (not shown). All slides were imaged using identical microscope settings and image processing techniques to ensure that miR-29a and miR-29b localization patterns could be directly compared.

### Example 1

### miR-29 Cell Cycle Stage-Specific Expression Pattern and Accumulation

Expression Patterns of miR-29 Paralogs

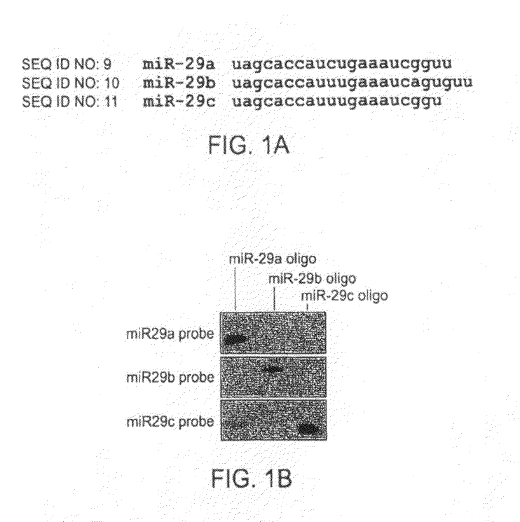

Prior examination of cell-cycle stage-specific miRNA expression patterns with a previously described oligonucleotide array (K. A. O'Donnell, E. A. Wentzel, K. I. Zeller, C. V. Dang, J. T. Mendell, Nature 435, 839 (2005)) revealed substantial accumulation of miR-29 in mitotic HeLa cells (9). There are three human miR-29 paralogs: miR-29a, miR-29b, and miR-29c (FIG. 1A). The results of a highly specific Northern blot assay are shown in (FIG. 1B). The Northern Blot demonstrated that each miR-29 paralogue exhibits a distinct expression pattern. miR-29a is constitutively expressed in all cell-cycle phases, miR-29b is present at low levels except in mitotic cells, and miR-29c is not detectable (FIG. 2A). Human miR-29 family members are encoded by the miR-29b-1/miR-29a cluster and the miR-29b-2/miR-29c cluster (FIG. 2B). A fragment encompassing the miR-29b-1/miR-29a cluster was amplified by reverse transcription polymerase chain reaction (RT-PCR) after small interfering RNA (siRNA)-mediated inhibition of Drosha (which performs the first step in miRNA processing), demonstrating that these miRNAs are cotranscribed as a polycistronic primary transcript. In contrast, neither the miR-29b-2/miR-29c cluster primary transcript nor mature miR-29c was detected by RT-PCR or Northern blotting. These results indicate that miR-29b likely derives exclusively from the miR-29b-1/miR-29a cluster in HeLa cells. Moreover, given that miR-29a and miR-29b are co transcribed and miR-29a is constitutively expressed, a posttranscriptional mechanism must be functioning to prevent the accumulation of miR-29b in all cell-cycle phases except mitosis. Posttranscriptional regulation of miRNA abundance could occur at the level of miRNA maturation or stability. miRNA maturation is a two-step process involving sequential cleavages by Drosha and Dicer (B. R. Cullen, Mol. Cell 16, 861 (2004); V. N. Kim, Nat. Rev. Mol. Cell. Biol. 6, 376 (2005)).

miR-29 Processing

To assess Drosha processing of miR-29b-1, RT-PCR was performed with an amplicon spanning the premiRNA hairpin (FIG. 2C, amplicon 1). As expected, an RT-PCR product was observed only after siRNA-mediated depletion of Drosha. Failure to amplify this region in the presence of Drosha activity was not due to a general instability of the primary transcript because an amplicon that did not span a Drosha cleavage site (FIG. 2C, amplicon 2) produced a product with control [luciferase (Luc)] or Drosha siRNA treatment. Two lines of evidence also suggest efficient processing of miR-29b by Dicer. First, an accumulation of pre-miR-29b, a species that frequently accumulates when Dicer processing is blocked, was never observed (J. M. Cummins et al., Proc. Natl. Acad. Sci. U.S.A. 103, 3687 (2006)). Second, a fully processed synthetic siRNA-like miR-29b duplex (si-miR-29b) mimicked the endogenously expressed miRNA and accumulated in cells arrested in mitosis (FIG. 3). Thus, the mitotic accumulation of miR-29b occurs after it is fully processed and therefore is most likely due to enhanced stability of the miRNA in this cell cycle phase.

Measurement of miRNA Stability in Cycling and Mitotic Cells

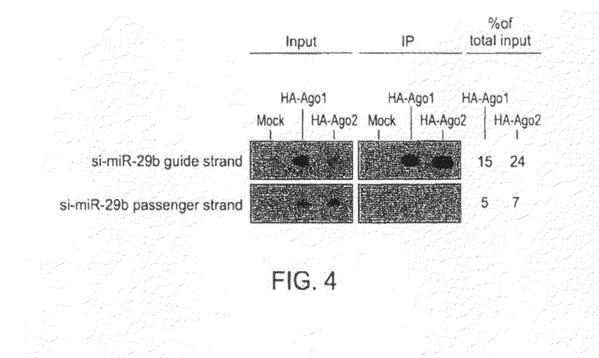

In order to measure miRNA stability in cycling and mitotic cells, a pulse-chase strategy was used with synthetic miRNA duplexes (si-miRNAs). Multiple lines of evidence indicate that si-miR-29b accurately recapitulates the behavior of the endogenous miRNA. First, as mentioned above, si-miR-29b exhibits mitotic accumulation like the endogenous miRNA (FIG. 3). Second, immunoprecipitation experiments with human Argonaute 1 or 2. demonstrate that si-miR-29b duplexes are appropriately loaded into the RNA-induced silencing complex (FIG. 4). Cells were pulsed with si-miRNAs, and the fraction that remained was monitored over time (FIG. 2D). si-miR-29b degraded rapidly in cycling cells but was stable in mitotic cells. As expected, si-miR-29a degraded slowly under both tested conditions.

In these studies, nocodazole treatment was used to obtain cells arrested in mitosis. Several experiments were performed to demonstrate that nocodazole-induced accumulation of miR-29b was a result of mitotic arrest rather than nonspecific perturbation of microtubule dynamics. To rule out the possibility that accumulation of miR-29b was related to perturbation of microtubule dynamics by nocodazole independent of mitotic arrest, HeLa cells synchronized at G1/S were released into nocodazole-containing media. miR-29b accumulated only when cells entered mitosis despite being exposed to nocodazole throughout a prolonged time-course (FIG. 5A). Furthermore, only nocodazole-treated cells that detached and entered mitosis, but not those that remained adherent, showed miR-29b accumulation (FIG. 5B). Elevated levels of miR-29b were also observed after treatment of cells with other microtubule poisons that have diverse effects on microtubule dynamics but are common in their ability to arrest cells in mitosis (FIG. 5C) (D. Leaman et al., Cell 121, 1097 (2005)). Finally, it was demonstrated that accumulation of miR-29b was not related to apoptosis, which is known to occur when cells are arrested at the spindle checkpoint (M. Castedo et al., Oncogene 23, 2825 (2004)). Treatment with cisplatin or camptothecin induced apoptosis without mitotic arrest and did not cause miR-29b accumulation (FIG. 5D). It was additionally demonstrated that the accumulation of miR-29b was not related to apoptosis, which is known to occur when cells are arrested at the spindle checkpoint (FIG. 5D) (M. Castedo et al., Oncogene 23, 2825 (2004)).

### Example 2

### miR-29 Subcellular Localization

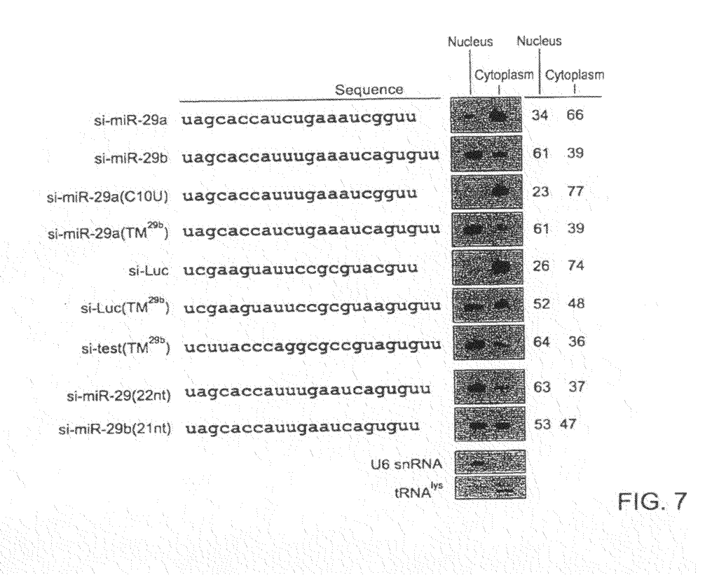

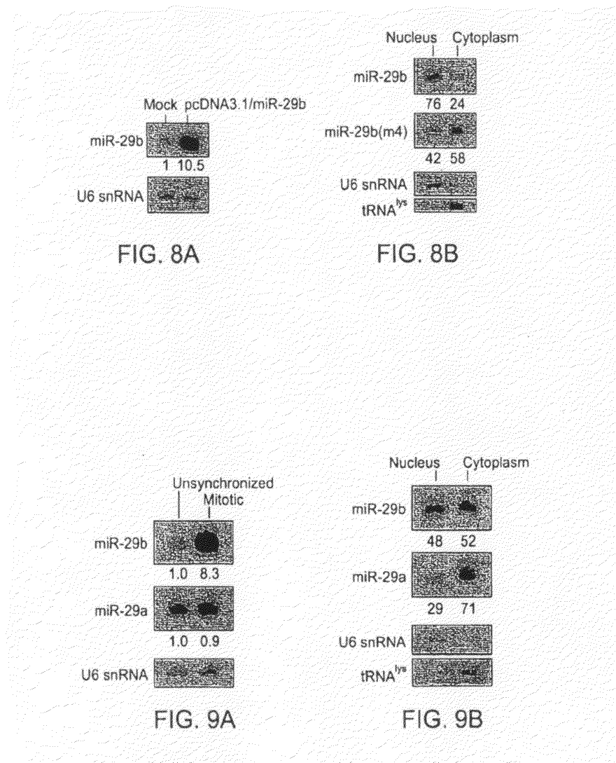

miR-29a and all previously studied animal miRNAs are predominantly cytoplasmic (FIG. 6A) (M. L. Whitfield et al., Mol. Cell. Biol. 20, 4188 (2000); P. Leeds, S. W. Peltz, A. Jacobson, M. R. Culbertson, Genes Dev. 5, 2303 (1991).). Because disassembly of the nuclear membrane distinguishes mitosis from other cell cycle phases, the subcellular localization of miR-29b was examined next. Cellular fractionation revealed that miR-29b (which exists at low but detectable levels in cycling cells) is predominantly nuclear. Synthetic si-miR-29a and si-miR-29b exhibited the identical localization pattern as their native counterparts, as shown in FIG. 7. A plasmid expressing a fragment of the miR-29b-1 primary transcript also produced mature miR-29b that trafficked to the nucleus (FIG. 8). Fluorescence in situ hybridization and confocal microscopy demonstrated punctate cytoplasmic localization of transfected si-miR-29a or si-miR-29b duplexes in a pattern that is consistent with other published reports (FIG. 6B) (P. Leeds, I. M. Wood, B. S. Lee, M. R. Culbertson, Mol. Cell. Biol. 12, 2165 (1992)). In addition, substantially more punctate and diffuse intranuclear staining was visible for si-miR-29b. These data indicate that miR-29b is imported into the nucleus in cycling cells. miR-29b also shows mitotic accumulation and nuclear enrichment in murine NIH 3T3 cells (FIG. 9), demonstrating the conservation of this pathway in other mammalian cell lines.

Measurement of miRNA Decay Rates Using si-miRNA Duplexes

It is difficult to obtain a reliable measurement of the half-life of endogenously expressed miR-29b due to both its low abundance in cycling cells and the highly toxic effect of combining pharmacologic inhibition of transcription and mitotic arrest. Nevertheless, several lines of evidence demonstrate that si-miR-29b recapitulates the behavior of the endogenous miRNA. First, si-miR-29b exhibits mitotic accumulation like the endogenous miRNA (FIG. 3). Second, immunoprecipitation experiments with hAgo1 or hAgo2 demonstrate that si-miR-29b duplexes are appropriately loaded into RISC with preservation of the endogenous strand selection bias (FIG. 4). Third, these experiments are assaying the unwound duplexes since the si-miR-29b guide strand was predominantly nuclear and the si-miR-29b passenger strand was predominantly cytoplasmic (FIG. 12).

### Example 3

### miR-29 Hexanucleotide 3′ Terminal Motifs

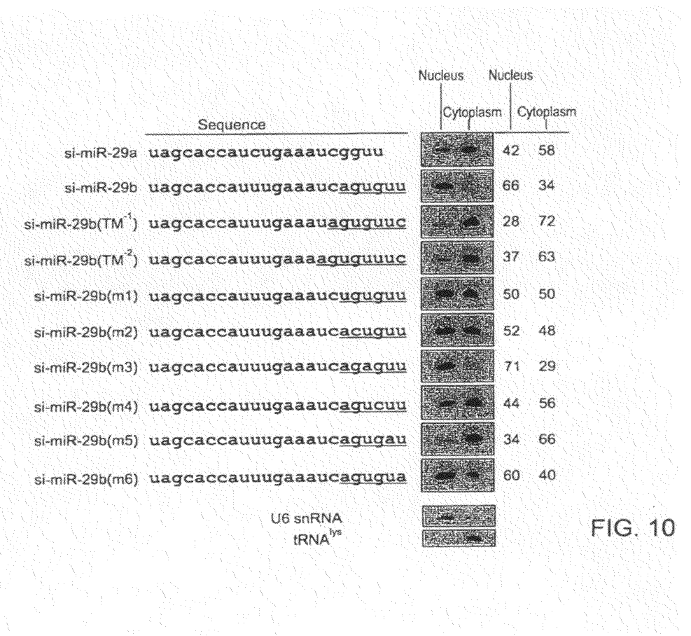

Because si-miR-29a and si-miR-29b behave exactly like the endogenously expressed miRNAs, sequence elements contained within the fully processed molecules must specify their distinct localization patterns. A uridine at nucleotide 10 and a distinctive hexanucleotide 3′ terminal motif (AGUGUU) distinguish miR-29b from miR-29a (FIG. 3). Variant si-miR-29a duplexes containing a uridine at position 10 [si-miR-29a(C10U)] were cytoplasmic, whereas si-miR-29a duplexes tagged with the miR-29b 3′ terminal motif [si-miR-29a(TM29b)] were enriched in the nucleus (FIG. 7). To determine whether this motif could confer nuclear localization to an unrelated sequence, its ability to direct nuclear import of a functional siRNA directed against luciferase was assessed (Y. Lee et al., Nature 425, 415 (2003). Addition of the miR-29b terminal motif was sufficient to direct nuclear enrichment [FIG. 7, si-Luc(TM29b)]. An additional unrelated siRNA ending in AGUGUU was tested and found to be highly enriched in the nuclear compartment [FIG. 7, si-test(TM29b)]. Internal deletions introduced into si-miR-29b demonstrated that the motif can specify nuclear import of 22- or 21-nucleotide miRNAs (FIG. 7). However, the motif must be at the 3′ terminus of the miRNA in order to function [FIG. 10, si-miR-29b(TM-1) and si-miR-29b(TM-2)]. Finally, the consequences of transversion mutations at each position of the element were determined. Mutations at four positions substantially reduced the nuclear targeting efficiency (FIG. 10). ThemiR-29b terminal motif or a relaxed consensus supported by these mutagenesis studies (AGNGUN, where N is any nucleotide) is not present in other miRNAs that are conserved throughout the mammalian radiation (see below). Therefore, the mir-29b terminal motif appears to be rarely, if ever, used by other mammalian miRNAs for nuclear localization.

Other Natural miRNAs with the miR-29b Terminal Motif

Three miRNAs within the large primate-specific miRNA cluster on chromosome 19 have been reported to end in AGUGUU (miR-517a, miR-517b, and miR-522) (I. Bentwich et al., Nat. Genet. 37, 766 (2005)). Nevertheless, miR-517 family members undergo heterogeneous 3′ end processing, so it is not clear what subpopulation of mature miR-517 molecules end in this motif. Because variable 3′ ends were not described for miR-522, the nuclear and cytoplasmic abundance of this miRNA were measured. However, northern blotting did not detect expression of the mature miR-522 species in primary placental cultures or after heterologous expression in HeLa cells, suggesting that it may not be a bona fide miRNA. Thus, one conclusion is that the mir-29b terminal motif as defined herein, is rarely, if ever, used by other mammalian miRNAs for nuclear localization.

### Example 4

### miR-29 Imported into the Nucleus Does Not Undergo Accelerated Turnover

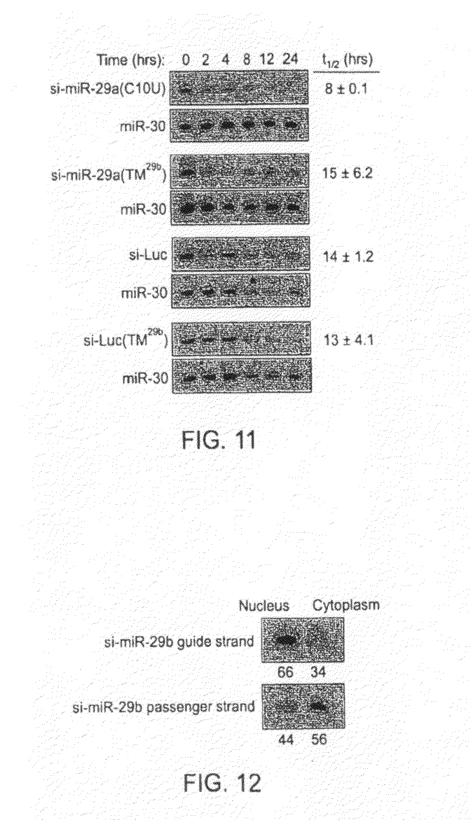

Although miR-29b undergoes rapid decay, accelerated turnover does not appear to be a general feature of small RNAs that are imported into the nucleus. The addition of the miR-29b terminal motif did not lead to the accelerated decay of miR-29a or Luc siRNA (FIG. 11). The introduction of the C-to-U substitution at position 10 into miR-29a [si-miR-29a(C10U)] resulted in a shorter half-life but did not fully recapitulate the rapid decay observed for miR-29b (FIG. 11). These findings indicate that the miR-29b terminal motif may be useful for designing stable siRNAs or miRNAs that are highly enriched in the nucleus. These RNAs may prove useful for the manipulation of nuclear steps in gene expression. Indeed, it is possible that the natural function of miR-29b is to regulate transcription or splicing of target transcripts, rather than the canonical translation regulatory functions that are ascribed to other miRNAs.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

### LITERATURE CITED

1. M. A. Valencia-Sanchez, J. Liu, G. J. Hannon, R. Parker, Genes Dev. 20, 515 (2006).
2. T. Du, P. D. Zamore, Development 132, 4645 (2005).
3. J. Brennecke, A. Stark, R. B. Russell, S. M. Cohen, PLoS Biol. 3, e85 (2005).
4. E. C. Lai, Genome Biol. 5, 115 (2004).
5. B. P. Lewis, C. B. Burge, D. P. Bartel, Cell 120, 15 (2005).
6. D. Leaman et al., Cell 121, 1097 (2005).
7. A. L. Abbott et al., Dev. Cell 9, 403 (2005).
8. K. A. O'Donnell, E. A. Wentzel, K. I. Zeller, C. V. Dang, J. T. Mendell, Nature 435, 839 (2005).
9. Materials and methods are available as supporting material on Science Online.
10. B. R. Cullen, Mol. Cell 16, 861 (2004).
11. V. N. Kim, Nat. Rev. Mol. Cell. Biol. 6, 376 (2005).
12. J. M. Cummins et al., Proc. Natl. Acad. Sci. U.S.A. 103, 3687 (2006).
13. M. Castedo et al., Oncogene 23, 2825 (2004).
14. M. L. Whitfield et al., Mol. Cell. Biol. 20, 4188 (2000).
15. P. Leeds, S. W. Peltz, A. Jacobson, M. R. Culbertson, Genes Deli. 5, 2303 (1991).
16. P. Leeds, J. M. Wood, B. S. Lee, M. R. Culbertson, Mol. Cell. Biol. 12, 2165 (1992).
17. Y. Lee et al., Nature 425, 415 (2003).
18. S. M. Elbashir et al., Nature 411, 494 (2001).
19. J. Zhou, D. Panda, J. W. Landen, L. Wilson, H. C. Joshi, J. Biol. Chem. 277, 17200 (2002).
20. M. Castedo et al., Oncogene 23, 2825 (2004).
21. I. Bentwich et al., Nat. Genet. 37, 766 (2005).

1. An isolated nucleotide motif comprising a localization element for an RNA or protein.

2. The nucleotide motif comprising a localization element of claim 1, wherein the localization element is attached to the 3′ terminus of the RNA.

3. The nucleotide motif comprising a localization element of claim 1, wherein the localization element is a hexanucleotide motif.

4. The nucleotide motif comprising a localization element of claim 1, wherein the localization element directs nuclear localization of the RNA or protein.

5. The nucleotide motif comprising a localization element of claim 1, wherein the localization element promotes nuclear import of the RNA or protein.

6. The nucleotide motif comprising a localization element of claim 1, wherein the localization element increases nuclear accumulation of the RNA.

7. The nucleotide motif comprising a localization element of claim 1, wherein the localization element silences expression of the RNA target sequence.

8. The nucleotide motif comprising a localization element of claim 1, wherein the RNA is a small RNA.

9. The nucleotide motif comprising a localization element of claim 8, wherein the small RNA is selected from a small interfering RNA (siRNA), a microRNA (miRNA), a Piwi-interacting RNA (piRNA), or a repeat associated siRNA (rasiRNA).

10. The nucleotide motif of claim 8, wherein the small RNA is modified.

11. The nucleotide motif of claim 10, wherein the modification is a chemical modification.

12. The nucleotide motif of claim 11, wherein the modification comprises linking the small RNA to an agent.

13. The nucleotide motif of claim 12, wherein the agent is selected from one or more of an antibody, an aptamer or cholesterol.

14. The nucleotide motif comprising a localization element of claim 1 wherein the localization element is selected from the group consisting of: SEQ ID NO: 1 (AGUGUU), SEQ ID NO: 2 (UGUGUU), SEQ ID NO: 3 (ACUGUU), SEQ ID NO: 4 (AGAGUU), SEQ ID NO: 5 (AGUCUU), SEQ ID NO: 6 (AGUGAU) and SEQ ID NO: 7 (AGUGUA).

15. The nucleotide motif comprising a localization element of claim 1, wherein the localization element comprises SEQ ID NO: 8 (AGNGUN), where N is any nucleotide.

16. An isolated RNA molecule comprising a first segment comprising a nucleotide motif comprising a localization element, and a second segment comprising a region of nucleotides adjacent to the localization element.

17. The RNA molecule of claim 16, wherein the first segment and the second segment comprise a double-stranded region of nucleotides about 15-80 nucleotides in length.

18. The RNA molecule of claim 16, wherein the nucleotide motif comprising a localization element is adjacent to the 3′ terminus of the second segment.

19. The RNA molecule of claim 16, wherein the second segment double-stranded region of nucleotides adjacent to the first segment double-stranded region of nucleotides provides specificity for the RNA to a target gene.

20. The RNA molecule of claim 16, wherein the nucleotide motif comprising a localization element is located at the 3′ terminus of the double-stranded nucleotide region.

21. The RNA molecule of claim 16, wherein the first segment and the second segment comprising a double-stranded region of nucleotides are annealed such that there is a 2 nucleotide 3′ overhang.

22. The RNA molecule of claim 16, wherein the RNA is a small RNA.

23. The RNA molecule of claim 16, wherein the small RNA is selected from a small interfering RNA (siRNA), a microRNA (miRNA), a Piwi-interacting RNA (piRNA), or a repeat associated siRNA (rasiRNA).

24. The RNA molecule of claim 22, wherein the small RNA is modified.

25. The RNA molecule of claim 24, wherein the modification is a chemical modification.

26. The RNA molecule of claim 24, wherein the modification comprises linking the small RNA to an agent.

27. The RNA molecule of claim 26, wherein the agent is selected from one or more of an antibody, an aptamer, or cholesterol.

28. The RNA molecule of claim 16, wherein the second segment comprises a region of about 15-80 nucleotides in length comprising a sense RNA strand and an antisense RNA strand that forms an RNA duplex that is approximately 20-80 nucleotides in length

29. The RNA molecule of claim 28, wherein the sense and antisense RNA strands that form an RNA duplex are covalently linked by a single-stranded hairpin.

30. The RNA molecule of claim 16, wherein the RNA molecule is targeted to any stretch of approximately 20-35 contiguous nucleotides in a target mRNA sequence.

31. An isolated small RNA molecule comprising a first and second segment comprising a sense RNA strand and an antisense RNA strand, wherein the sense and the antisense RNA strands form an RNA duplex, and wherein the small RNA molecule is targeted to any stretch of approximately 20-35 contiguous nucleotides in a target mRNA sequence, and wherein the first segment comprises a nucleotide motif comprising a localization element.

32. The small RNA molecule of claim 31, wherein the second segment comprising a sense RNA strand and an antisense RNA strand is about 15-80 nucleotides in length adjacent to the nucleotide sequence.

33. The small RNA molecule of claim 31, wherein the siRNA or miRNA molecule comprises two strands, and at least 1 strand has a 3′ overhang of about 1 to about 6 nucleotides in length.

34. The small RNA molecule of claim 31, wherein the small RNA is selected from a small interfering RNA (siRNA), a microRNA (miRNA), a Piwi-interacting RNA (piRNA), or a repeat associated siRNA (rasiRNA).

35. The RNA molecule of claim 16, wherein the localization element is selected from the group consisting of: SEQ ID NO: 1 (AGUGUU), SEQ ID NO: 2 (UGUGUU), SEQ ID NO: 3 (ACUGUU), SEQ ID NO: 4 (AGAGUU), SEQ ID NO: 5 (AGUCUU), SEQ ID NO: 6 (AGUGAU) and SEQ ID NO: 7 (AGUGUA).

36. The RNA molecule of claim 16, wherein the localization element comprises SEQ ID NO: 8 (AGNGUN), where N is any nucleotide.

37. The RNA molecule of claim 16, wherein the RNA molecule or the small RNA molecule is capable of inhibiting the expression of a target gene in a cell.

38. The RNA molecule of claim 16, wherein the gene silencing is transcriptional gene silencing.

39. The RNA molecule of claim 16, wherein the gene silencing is post-translational gene silencing.

40. The RNA molecule of claim 16, wherein the target gene comprises one or more of an endogenous cellular gene, an exogenous gene or a viral gene.

41. The RNA molecule of claim 16, wherein the target gene is of mammalian origin.

42. The RNA molecule of claim, wherein the target gene is expressed in a phenotypically normal cell.

43. The RNA molecule of claim 16, wherein the target gene is expressed in a diseased cell.

44. (canceled)

45. The RNA molecule of claim 16, wherein the expression of the target gene is inhibited by at least 50%, 60%, 70%, 75%, 80%, 85%, 90%, 95%, or more by the small RNA molecule.

46. (canceled)

47. (canceled)

48. (canceled)

49. (canceled)

50. (canceled)

51. A method for preventing or treating a disease or disorder, comprising, administering to a subject in need thereof a pharmaceutical composition comprising a therapeutically effective amount of an RNA molecule according to claim 16 or a small RNA molecule according to claim 31, thereby preventing or treating a disease or disorder.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. A pharmaceutical composition comprising as an active ingredient a RNA molecule according to claim 16, and a pharmaceutically acceptable carrier.

70. An expression vector capable of coding for the RNA molecule according to claim 16.

71. A pharmaceutical composition comprising the vector of claim 70.

72. (canceled)

73. (canceled)

74. (canceled)

75. (canceled)

76. (canceled)

77. (canceled)

78. A kit comprising the nucleotide motif comprising a localization element of any one of claim 1, and instructions for use.

79. A kit comprising the RNA molecule of claim 16, and instructions for use.

80. (canceled)


SEQUENCE LISTING (77 sequences)

SEQ ID NO: 1 - RNA, 6 nt, Artificial Sequence
  Description of Artificial Sequence Synthetic oligonucleotide
aguguu

SEQ ID NO: 2 - RNA, 6 nt, Artificial Sequence
  Description of Artificial Sequence Synthetic oligonucleotide
uguguu

SEQ ID NO: 3 - RNA, 6 nt, Artificial Sequence
  Description of Artificial Sequence Synthetic oligonucleotide
acuguu

SEQ ID NO: 4 - RNA, 6 nt, Artificial Sequence
  Description of Artificial Sequence Synthetic oligonucleotide
agaguu

SEQ ID NO: 5 - RNA, 6 nt, Artificial Sequence
  Description of Artificial Sequence Synthetic oligonucleotide
agucuu

SEQ ID NO: 6 - RNA, 6 nt, Artificial Sequence
  Description of Artificial Sequence Synthetic oligonucleotide
agugau

SEQ ID NO: 7 - RNA, 6 nt, Artificial Sequence
  Description of Artificial Sequence Synthetic oligonucleotide
agugua

SEQ ID NO: 8 - RNA, 6 nt, Artificial Sequence
  Description of Artificial Sequence Synthetic oligonucleotide
agngun

SEQ ID NO: 9 - RNA, 21 nt, Artificial Sequence
  Description of Artificial Sequence Synthetic oligonucleotide
uagcaccaucugaaaucgguu

SEQ ID NO: 10 - RNA, 23 nt, Artificial Sequence
  Description of Artificial Sequence Synthetic oligonucleotide
uagcaccauuugaaaucaguguu

SEQ ID NO: 11 - RNA, 20 nt, Artificial Sequence
  Description of Artificial Sequence Synthetic oligonucleotide
uagcaccauuugaaaucggu

SEQ ID NO: 12 - DNA, 27 bp, Artificial Sequence
  Description of Artificial Sequence Synthetic primer
ttgtcttgggtttattgtaagagagca

SEQ ID NO: 13 - DNA, 19 bp, Artificial Sequence
  Description of Artificial Sequence Synthetic primer
agggcagaggctgggtctt

SEQ ID NO: 14 - DNA, 22 bp, Artificial Sequence
  Description of Artificial Sequence Synthetic primer
gacatctgtcaccccattgatc

SEQ ID NO: 15 - DNA, 23 bp, Artificial Sequence
  Description of Artificial Sequence Synthetic primer
caacggtcaccaatacatttcct

SEQ ID NO: 16 - DNA, 21 bp, Artificial Sequence
  Description of Artificial Sequence Synthetic primer
ccatccatcttccaggaaacc

SEQ ID NO: 17 - DNA, 22 bp, Artificial Sequence
  Description of Artificial Sequence Synthetic primer
tctgcagtcttggaagcttgac

SEQ ID NO: 18 - DNA, 24 bp, Artificial Sequence
  Description of Artificial Sequence Synthetic primer
aacagcaggatggctgtgagaacc

SEQ ID NO: 19 - DNA, 23 bp, Artificial Sequence
  Description of Artificial Sequence Synthetic primer
acgaccttctgtgaccccttaga

SEQ ID NO: 20 - DNA, 25 bp, Artificial Sequence
  Description of Artificial Sequence Synthetic primer
acttcttttgctgttggtagtgcag

SEQ ID NO: 21 - DNA, 24 bp, Artificial Sequence
  Description of Artificial Sequence Synthetic primer
aacccaggcacaatggaatgagtc

SEQ ID NO: 22 - DNA, 32 bp, Artificial Sequence
  Description of Artificial Sequence Synthetic primer
ataccggctagcgcatgctctcccatcaataa

SEQ ID NO: 23 - DNA, 31 bp, Artificial Sequence
  Description of Artificial Sequence Synthetic primer
aggctcgagtgaagcaaggaactggagatgg

SEQ ID NO: 24 - DNA, 32 bp, Artificial Sequence
  Description of Artificial Sequence Synthetic primer
ataccgctcgaggacctgactgccatttgtga

SEQ ID NO: 25 - DNA, 30 bp, Artificial Sequence
  Description of Artificial Sequence Synthetic primer
aggctcgagactccagtttgggcagcagac

SEQ ID NO: 26 - DNA, 21 bp, Artificial Sequence
  Description of Artificial Sequence Synthetic oligonucleotide
aaccgatttcagatggtgcta

SEQ ID NO: 27 - DNA, 23 bp, Artificial Sequence
  Description of Artificial Sequence Synthetic oligonucleotide
aacactgatttcaaatggtgcta

SEQ ID NO: 28 - DNA, 23 bp, Artificial Sequence
  Description of Artificial Sequence Synthetic oligonucleotide
gctgagagtgtaggatgtttaca

SEQ ID NO: 29 - DNA, 22 bp, Artificial Sequence
  Description of Artificial Sequence Synthetic oligonucleotide
tcaacatcagtctgataagcta

SEQ ID NO: 30 - DNA, 37 bp, Artificial Sequence
  Description of Artificial Sequence Synthetic oligonucleotide
gaatttgcgtgtcatccttgcgcaggggccatgctaa

SEQ ID NO: 31 - DNA, 30 bp, Artificial Sequence
  Description of Artificial Sequence Synthetic oligonucleotide
ctgatgctctaccgactgagctatccgggc

SEQ ID NO: 32 - RNA, 21 nt, Artificial Sequence
  Description of Artificial Sequence Synthetic oligonucleotide
ccgauuucagauggugcuauu

SEQ ID NO: 33 - RNA, 23 nt, Artificial Sequence
  Description of Artificial Sequence Synthetic oligonucleotide
cacugauuucaaauggugcuauu

SEQ ID NO: 34 - RNA, 21 nt, Artificial Sequence
  Description of Artificial Sequence Synthetic oligonucleotide
ccgauuucaaauggugcuauu

SEQ ID NO: 35 - RNA, 21 nt, Artificial Sequence
  Description of Artificial Sequence Synthetic oligonucleotide
uagcaccauuugaaaucgguu

SEQ ID NO: 36 - RNA, 23 nt, Artificial Sequence
  Description of Artificial Sequence Synthetic oligonucleotide
cacugauuucagauggugcuauu

SEQ ID NO: 37 - RNA, 23 nt, Artificial Sequence
  Description of Artificial Sequence Synthetic oligonucleotide
uagcaccaucugaaaucaguguu

SEQ ID NO: 38 - RNA, 21 nt, Artificial Sequence
  Description of Artificial Sequence Synthetic oligonucleotide
cguacgcggaauacuucgauu

SEQ ID NO: 39 - RNA, 21 nt, Artificial Sequence
  Description of Artificial Sequence Synthetic oligonucleotide
ucgaaguauuccgcguacguu

SEQ ID NO: 40 - RNA, 23 nt, Artificial Sequence
  Description of Artificial Sequence Synthetic oligonucleotide
cacuuacgcggaauacuucgauu

SEQ ID NO: 41 - RNA, 23 nt, Artificial Sequence
  Description of Artificial Sequence Synthetic oligonucleotide
ucgaaguauuccgcguaaguguu

SEQ ID NO: 42 - RNA, 23 nt, Artificial Sequence
  Description of Artificial Sequence Synthetic oligonucleotide
cacuacggcgccuggguaagauu

SEQ ID NO: 43 - RNA, 23 nt, Artificial Sequence
  Description of Artificial Sequence Synthetic oligonucleotide
ucuuacccaggcgccguaguguu

SEQ ID NO: 44 - RNA, 22 nt, Artificial Sequence
  Description of Artificial Sequence Synthetic oligonucleotide
cacugauucaaauggugcuauu

SEQ ID NO: 45 - RNA, 22 nt, Artificial Sequence
  Description of Artificial Sequence Synthetic oligonucleotide
uagcaccauuugaaucaguguu

SEQ ID NO: 46 - RNA, 21 nt, Artificial Sequence
  Description of Artificial Sequence Synthetic oligonucleotide
cacugauucaauggugcuauu

SEQ ID NO: 47 - RNA, 21 nt, Artificial Sequence
  Description of Artificial Sequence Synthetic oligonucleotide
uagcaccauugaaucaguguu

SEQ ID NO: 48 - RNA, 23 nt, Artificial Sequence
  Description of Artificial Sequence Synthetic oligonucleotide
acacuauuucaaauggugcuauc

SEQ ID NO: 49 - RNA, 23 nt, Artificial Sequence
  Description of Artificial Sequence Synthetic oligonucleotide
uagcaccauuugaaauaguguuc

SEQ ID NO: 50 - RNA, 23 nt, Artificial Sequence
  Description of Artificial Sequence Synthetic oligonucleotide
aacacuuuucaaauggugcuauc

SEQ ID NO: 51 - RNA, 23 nt, Artificial Sequence
  Description of Artificial Sequence Synthetic oligonucleotide
uagcaccauuugaaaaguguuuc

SEQ ID NO: 52 - RNA, 23 nt, Artificial Sequence
  Description of Artificial Sequence Synthetic oligonucleotide
cacagauuucaaauggugcuauu

SEQ ID NO: 53 - RNA, 23 nt, Artificial Sequence
  Description of Artificial Sequence Synthetic oligonucleotide
uagcaccauuugaaaucuguguu

SEQ ID NO: 54 - RNA, 23 nt, Artificial Sequence
  Description of Artificial Sequence Synthetic oligonucleotide
cagugauuucaaauggugcuauu

SEQ ID NO: 55 - RNA, 23 nt, Artificial Sequence
  Description of Artificial Sequence Synthetic oligonucleotide
uagcaccauuugaaaucacuguu

SEQ ID NO: 56 - RNA, 23 nt, Artificial Sequence
  Description of Artificial Sequence Synthetic oligonucleotide
cucugauuucaaauggugcuauu

SEQ ID NO: 57 - RNA, 23 nt, Artificial Sequence
  Description of Artificial Sequence Synthetic oligonucleotide
uagcaccauuugaaaucagaguu

SEQ ID NO: 58 - RNA, 23 nt, Artificial Sequence
  Description of Artificial Sequence Synthetic oligonucleotide
gacugauuucaaauggugcuauu

SEQ ID NO: 59 - RNA, 23 nt, Artificial Sequence
  Description of Artificial Sequence Synthetic oligonucleotide
uagcaccauuugaaaucagucuu

SEQ ID NO: 60 - RNA, 23 nt, Artificial Sequence
  Description of Artificial Sequence Synthetic oligonucleotide
uagcaccauuugaaaucagugau

SEQ ID NO: 61 - RNA, 23 nt, Artificial Sequence
  Description of Artificial Sequence Synthetic oligonucleotide
uagcaccauuugaaaucagugua

SEQ ID NO: 62 - DNA, 23 bp, Artificial Sequence
  Description of Artificial Sequence Synthetic oligonucleotide
aatagcaccatttgaaatcagtg

SEQ ID NO: 63 - DNA, 21 bp, Artificial Sequence
  Description of Artificial Sequence Synthetic oligonucleotide
aaccgatttcaaatggtgcta

SEQ ID NO: 64 - DNA, 23 bp, Artificial Sequence
  Description of Artificial Sequence Synthetic oligonucleotide
aacactgatttcagatggtgcta

SEQ ID NO: 65 - DNA, 21 bp, Artificial Sequence
  Description of Artificial Sequence Synthetic oligonucleotide
aacgtacgcggaatacttcga

SEQ ID NO: 66 - DNA, 23 bp, Artificial Sequence
  Description of Artificial Sequence Synthetic oligonucleotide
aacacttacgcggaatacttcga

SEQ ID NO: 67 - DNA, 23 bp, Artificial Sequence
  Description of Artificial Sequence Synthetic oligonucleotide
aacactacggcgcctgggtaaga

SEQ ID NO: 68 - DNA, 22 bp, Artificial Sequence
  Description of Artificial Sequence Synthetic oligonucleotide
aacactgattcaaatggtgcta

SEQ ID NO: 69 - DNA, 21 bp, Artificial Sequence
  Description of Artificial Sequence Synthetic oligonucleotide
aacactgattcaatggtgcta

SEQ ID NO: 70 - DNA, 23 bp, Artificial Sequence
  Description of Artificial Sequence Synthetic oligonucleotide
gaacactatttcaaatggtgcta

SEQ ID NO: 71 - DNA, 23 bp, Artificial Sequence
  Description of Artificial Sequence Synthetic oligonucleotide
gaaacacttttcaaatggtgcta

SEQ ID NO: 72 - DNA, 23 bp, Artificial Sequence
  Description of Artificial Sequence Synthetic oligonucleotide
aacacagatttcaaatggtgcta

SEQ ID NO: 73 - DNA, 23 bp, Artificial Sequence
  Description of Artificial Sequence Synthetic oligonucleotide
aacagtgatttcaaatggtgcta

SEQ ID NO: 74 - DNA, 23 bp, Artificial Sequence
  Description of Artificial Sequence Synthetic oligonucleotide
aactctgatttcaaatggtgcta

SEQ ID NO: 75 - DNA, 23 bp, Artificial Sequence
  Description of Artificial Sequence Synthetic oligonucleotide
aagactgatttcaaatggtgcta

SEQ ID NO: 76 - DNA, 23 bp, Artificial Sequence
  Description of Artificial Sequence Synthetic oligonucleotide
atcactgatttcaaatggtgcta

SEQ ID NO: 77 - DNA, 23 bp, Artificial Sequence
  Description of Artificial Sequence Synthetic oligonucleotide
tacactgatttcaaatggtgcta